(19) United States
(12) Patent Application Publication
(10) Pub. No.: US 2022/0044764 A1
(43) Pub. Date: Feb. 10, 2022

(54) TREATMENT OF CANCER BY RISK STRATIFICATION OF PATIENTS BASED ON COMORDIDITIES

(71) Applicants: Balaji Narayana Reddy, Mt Laurel, NJ (US); Madanika Subhash, Mt Laurel, NJ (US)

(72) Inventors: Balaji Narayana Reddy, Mt Laurel, NJ (US); Madanika Subhash, Mt Laurel, NJ (US)

(21) Appl. No.: 17/451,482

(22) Filed: Oct. 19, 2021

Related U.S. Application Data
(63) Continuation-in-part of application No. 15/635,216, filed on Jun. 28, 2017.

(51) Int. Cl. G16B 40/00; G16B 50/00; G16H 50/50

(52) U.S. Cl. CPC G16B 40/00; G16H 50/50; G16B 50/00

(57) ABSTRACT

A method is provided of identifying genes associated with poor clinical outcomes for a particular cancer. Genes encoding a comorbidity associated with a poor clinical outcomes for a particular cancer are identified in a cohort of patients with the cancer. Gene alterations and mutations associated with the comorbidities are determined. Gene expression level associated with the comorbidities are normalized against the expression level of a reference set of RNA transcripts in patients without the cancer or comorbidity. A statistical analysis compares the pathological gene expression level with normal gene expression level to create a database of statistically significant genes wherein the expression level of the abnormal genes is negatively associated with worse outcomes for the particular cancer. The method can be used to stage cancer, estimate prognosis, and for the design of therapeutic interventions by treating the comorbidity.

### CROSS REFERENCE TO RELATED APPLICATION

This patent application is a continuation-in-part of U.S. patent application Ser. No. 15/635,216, filed Jun. 28, 2017, the contents of which are incorporated by reference.

### FIELD OF THE INVENTION

The present invention identifies and ranks mutations and abnormalities in genes encoding comorbidities in cancer patients with disease severity. These mutations and abnormalities can be correlated to the diagnosis, prognosis, and treatment options for cancer patients.

### BACKGROUND

Medical comorbidities such as high blood pressure, diabetes, obesity, high cholesterol, smoking, alcohol consumption and others are known to be associated with the risk of developing long-term illnesses such as heart disease, eye disease, kidney disease among others. Evidence also points to the risk of developing certain kinds of cancer such as kidney cancer in patients suffering with these comorbidities. (1) Similarly, metabolic syndrome is associated with increased cancer risk. (2) Liver cancer, prostate cancer, thyroid cancer, pancreatic cancer, are among the types of cancer whose risk is increased by comorbidities. Thus, patients harboring these comorbidities may have higher risk of developing or harboring cancer despite an initial negative or an equivocal test or tests.

The initial diagnosis of cancer is usually based on a clinical suspicion or hunch by a physician. Tests are typically conducted to confirm a suspicion of cancer but are not always completely accurate. For example, a CT scan may show a kidney lesion, which is suspicious for cancer. The age and family history of a patient may point to a greater likelihood of cancer. In approximately 40% of the cases, these kidney lesions are non-cancerous. (3) In such clinical scenarios, additional tests such as a tissue biopsy, and/or genetic information may be of additional value, but their role is controversial. (4)

Following a cancer diagnosis, physicians have a number of treatment options available including different combinations of no treatment, delayed treatment, surveillance, surgical treatment, radiation, chemotherapeutic drugs or a combination of treatments, that collectively are characterized as the “standard of care” for any particular disease and patient. Additionally, a number of drugs or treatments that do not carry a label claim for a particular cancer but for which there is evidence of efficacy in that cancer are often used. The best likelihood of good treatment outcomes requires that patients be assigned to optimal available cancer treatment and that this assignment be made as quickly as possible following diagnosis.

Cancer can present in various stages. (5) An advanced stage cancer is usually worse in terms of severity of symptoms, including a poorer likelihood of survival than an early stage cancer. Therefore, physicians rely on various predictors to identify the risk of having advanced disease or identify those with greater risk of progressing to advanced disease. Identifying the patients who are less likely to progress is equally important. For example, African American ancestry is an important risk factor for more severe cancer related outcomes in patients with prostate cancer. Similarly, drinking excessive alcohol is associated with worse outcomes in patients with liver cancer. Also, smoking is related to worse outcomes in lung and bladder cancer. Genetic factors also predict risk profile. For example, male gender is associated with worse bladder cancer outcomes. Patients with alterations in certain genes are associated with worse outcomes than those without. Breast cancer patients with BRCA-1 and BRCA-2 gene alterations typically have worse outcomes than patients without these alterations.

Currently, clinical decisions in cancer patients do not always take into consideration the presence of comorbidities such as obesity, diabetes, high blood pressure, alcoholism, hormonal status, etc., in prognosticating the patient's cancer related outcomes. While some comorbidities such as smoking, alcoholism, obesity may appear unrelated to genetics, and more to do with an individual's choices, often the predisposition to and the outcomes of consuming alcohol, smoking, or gaining excess weight are genetically influenced. Certain comorbidities are also commonly genetically driven—such as obesity, diabetes, and high blood pressure. The progression of cancer may be driven by the interplay between the individual's choices, genetic predisposition for cancer, and any comorbidities which further influence the cancer outcomes. (6)

For example, the list of genes related to high blood pressure (hypertension), obesity and diabetes are ever increasing. Several sources, such as the website Online Mendelian Inheritance in Man® (OMIM®) (7) publishes genes along with the related scientific articles related to these genes. In this website, searching for the term high blood pressure yielded a set of genes attached in Table 1. Additional sources of hypertension genes include human-phenotype-ontology.github.io/. In this website, searching for the term high blood pressure yielded a set of genes attached in Table 2. Other source include an article recently published, which provides a method of predicting human hypertension genes. (8)

The understanding of the role of hypertension in cancer is well understood by knowing the pathophysiology of a cancer. Cancer grows by a method of new blood vessel formation, also called neovascularization. High blood pressure can also cause neovascularization leading to diseases such as hypertensive retinopathy. High blood pressure also induces changes in the blood vessels as a compensatory mechanism and induces changes in almost all organs of the body. High blood pressure is also attributed to improper electrolyte metabolism by the kidneys. Renal cell carcinoma is also known to cause high blood pressure. The cause of high blood pressure is multifactorial. It is also likely due to interaction between multiple genes.

### SUMMARY OF THE INVENTION

In an embodiment, this invention discloses a method of identifying individuals at risk of developing certain cancers, progression of cancers, regression of cancer following therapy, progression of cancers leading to metastatic disease, and progression of cancers leading to death, based on certain gene alterations or the level of gene expression in comorbidities.

In an embodiment, this invention discloses a method of identifying individuals at risk of developing certain cancers, progression of cancers, regression of cancer following therapy, progression to metastatic disease, and progression of cancer leading to death, based on the presence of factors leading to alterations in certain genes in comorbidities, leading to expression of these genes or presence of these gene products.

In an embodiment, this invention discloses a method of identifying individuals at risk of developing certain cancers, progression of cancers, regression of cancer following therapy, progression of cancers leading to metastatic disease, and progression of cancers leading to death, based on the presence of certain gene alterations related to high blood pressure.

In an embodiment, this invention discloses a method that incorporates any drugs developed to block the expression of comorbidity genes or the products of these genes alone or in combination with another chemotherapeutic agent or surgical therapy in preventing the progression of the disease.

In an embodiment, this invention discloses a method to detect the gene alterations, or their expression in comorbidities to cancer, which will help in identifying the risk of progression in individual patients.

This prognostic information may also be used to administer additional treatment or surgery with beneficial effect and outcome. This treatment may not always lead to a cure or a decrease in blood pressure but may target other mechanism(s) to alter or inhibit the cancer growth.

In the studies so far, the identification of cancer genes, and identifying the role of cancer genes thus identified were by comparing normal controls to cancer patients or comparing normal tissue to cancer tissue, without consideration to the comorbidities of the patient. Comorbidities are usually characterized as any medical condition(s) that the subject is at risk for, is diagnosed with, or treated for, as yet untreated, or with a genetic predisposition therefor. (9) Cancer patients could have these comorbidities either before the diagnosis of cancer, at the time of cancer diagnosis, or predisposed to develop it in the future. The comorbidities are identified by eliciting the relevant medical history from the subject(s), reviewing the medical records, performing diagnostic tests such as blood test, imaging tests, genetic tests to identify such genes, analyzing a sample of a tissue, reviewing the published literature for comorbidities associated with the cancer in question, or any method of diagnosis which is known to person skilled in the art of practicing medicine. The genes associated with comorbidities are usually but not always responsible for causing other medical conditions other than causing cancer in question.

None of the prior art discusses how to identify individuals at risk of developing certain cancers based on the presence of these comorbidities or based on the presence of gene alterations and/or gene expression associated with these comorbidities or based on the presence of factors leading to these gene alterations and/or gene expression.

None of the prior art discusses how to identify individuals at risk for faster progression of cancers based on the presence of these comorbidities or based on the presence of gene alterations and/or gene expression associated with these comorbidities or based on the presence of factors leading to these gene alterations and/or gene expression.

None of the prior art discusses how to identify individuals at risk of progression of cancers leading to metastatic disease based on the presence of these comorbidities or based on the presence of gene alterations and/or gene expression associated with these comorbidities or based on the presence of factors leading to these gene alterations and/or gene expression.

None of the prior art discusses how to identify individuals at risk of progression of cancers leading to death based on the presence of these comorbidities or based on the presence of gene alterations and/or gene expression associated with these comorbidities or based on the presence of factors leading to these gene alterations and/or gene expression.

### BRIEF DESCRIPTION OF THE DRAWINGS

FIG. 1 Gene alterations in subjects with renal cell carcinoma (clear cell type) of the TCGA, provisional data set comprising of 538 samples.

FIG. 2 Cancer specific survival of subjects with renal cell carcinoma (clear cell type), with alterations in the said genes.

FIG. 3. Gene alterations in subjects with prostate adenocarcinoma of the TCGA, provisional data set comprising of 499 samples.

FIG. 4. Cancer specific survival of subjects with prostate adenocarcinoma, with alterations in the said genes.

FIG. 5. Differential gene expression in patients with low (stage pT2 and lower) and high (stage pT3 and higher) stage renal cell carcinoma (clear cell type), taken from the UCSC Xena tool.

FIG. 6 is a flow chart of an embodiment of the inventive method.

FIG. 7 is a flow chart of an alternative embodiment of the inventive method.

### DETAILED DESCRIPTION

Due to the association of comorbidities with cancer occurrence and clinical outcome, a search for relevant mutations that lead to comorbidities associated cancer may be an important, yet unrecognized factor, in the diagnosis and treatment of cancer. This invention provides for improved diagnostic and treatment methods by addressing such mutations causing comorbidities associated with cancers.

One exemplary approach is to compile the top genes (with changes in the genes or their expression levels) in a particular cancer type after conducting appropriate statistical analysis using statistical methods known to person skilled in the art, then rank the genes associated with worse outcomes. One shortcoming of this approach is that unknown comorbidities, and gene alterations related to these comorbidities that may be driving the cancer, are not taken into consideration. With further analysis of cohorts of patients, such unknown comorbidities may be identified, and taking into consideration these comorbidities and the underlying genetic factors related to these comorbidities, future analyses for identifying predictors of cancer progression may overcome this limitation.

Several recent studies have published in cancer diagnosis and prognostication based on gene expression analysis. Recently several groups have published studies concerning classification of various cancer types by micro array gene expression analysis. (10) Classification of certain tumor types based on gene expression pattern has also been reported. (11) However, these studies do not provide the relationships of various comorbidities with the differentially expressed genes, and do not link the findings of treatment strategies in order to improve the clinical outcome of cancer therapy. Taking the genetic differences related to comorbidities into consideration is relevant because the genetic dysfunction of a comorbidity can also drive a cancer prognosis, and if a gene driving a comorbidity also drives cancer growth, the cancer outcome of such a patient is likely to be worse than the outcome where a gene underlying a comorbidity does not affect the cancer. The phrase “cancer outcome” means whether the cancer becomes more or less severe, for example, by a change in tumor size or a change in some other cancer marker indicating a more severe level of illness, including death of the patient that would not have occurred but for the cancer, or a less severe level of illness. To some extent, evaluating cancer outcomes means a prospective evaluation over weeks, months, or years to determine the progression of the disease.

Given that there are approximately 20,000 genes in humans, and the number of genes that can attain a statistically significant difference between the cohort of patients with good cancer outcomes compared to those with poor cancer outcomes is potentially large, it is difficult to achieve progress in developing effective strategies for identifying the genes primarily responsible for cancer prognosis and eventually to develop preventive, diagnostic and treatment methods. Thus, it is difficult to identify clinically relevant genes of interest in a large pool of statistically significant genes.

Moreover, pursuing all genes which are statistically identified to be different between groups of subjects with good or poor outcomes and their gene products as potential diagnostic or therapeutic targets is impractical. We therefore narrowed our genes of interest to the genes associated with certain comorbidities of interest. Comorbidities include any disease other than the medical condition being studied (a particular type of cancer in this case). (9) Examples include essential hypertension, obesity, type 1 diabetes, type 2 diabetes, chronic obstructive pulmonary disease, chronic kidney disease, coronary artery disease, stroke, various neurologic or psychiatric conditions including depression, dysthymia, anxiety disorders, bipolar disorders, drug abuse, alcohol abuse, smoking Parkinson's Disease, and Alzheimer's Disease. This is not intended to be a complete list of potentially relevant comorbidities. A more complete list is available on the International Statistical Classification of Diseases and Related Health Problems (ICD-10). (12)

For example, high blood pressure is associated with the development of various cancers. McLaughlin et al. reported an association in renal cell carcinoma with high blood pressure or from being on medication to treat high blood pressure. (13) The risk of developing high blood pressure is often determined by the genetic make-up of an individual, hormonal status, environmental factors that the patient is exposed to, and other factors. While the expression of these genes often is associated with high blood pressure, it may also be associated with other bodily functions and disease processes. One such untoward outcome is cancer.

By narrowing our focus to patients with medical conditions that lead to cancer, or medical conditions that lead to rapid progression of cancer, we can more effectively identify the genes (either alterations, or level of expression) associated with such medical conditions and identify their role in cancer related outcomes. Furthermore, it provides an opportunity to explore diagnostic, therapeutic and prognostic applications by using the identified genes.

Accordingly, an embodiment of this invention provides a method of identifying genes associated with poor clinical outcomes for a particular cancer, comprising a cohort (i.e., a group) of patients with the said cancer, identifying at least one comorbid medical condition, determining the gene alterations associated with at least one comorbidity, determining the gene expression level associated with at least one comorbidity, normalizing said gene expression level against the expression level of a reference set of RNA transcripts in patients without the cancer or comorbidity, performing a statistical analysis comparing the pathological gene expression level with normal gene expression level, and creating a database of statistically significant genes wherein the expression level of said genes encoding a comorbidity are associated with poor clinical outcomes for the particular cancer, wherein said genes are used to grade cancer outcomes for the particular cancer.

Identification of genes. In an embodiment, we identified genes relating to high blood pressure published in various sources. Several such sources include Online Mendelian Inheritance in Man® Online (OMIM®) (7), Human Phenotype Ontology (HPO) (14), The Cancer Genome Atlas (TCGA) (15), and the cBioPortal for Cancer Genomics (16). We used a statistical software to identify significant genes using methods known in the art. Two exemplary data sets are shown in Tables 1 and 2 appended hereto. Table 1 was obtained from Hsu et al. (17) Table 2 was obtained from the Human Phenotype Ontology database. (14)

The top genes which are determined to be of relevance were identified. Genes associated with high blood pressure, and closely linked to mTOR, PI3K, PTEN, and other known cancer genes are of particular interest. Such linked genes may cause a progression of cancer, resistance to cancer therapy, or cause a delay in diagnosis, thereby leading to worse outcomes. While any of these methods do not limit other ways to identify genes of interest in a particular patient or a group of patients, as the genes attributed to causing high blood pressure may be different in each individual, the genes of high blood pressure linked to known cancer genes likely relate to causing cancer progression. So, a highly expressed high blood pressure gene in an individual may be the target of a therapeutic intervention rather than a gene found to be most commonly expressed in patients with that particular cancer. The genes of interest can be detected using microarray techniques known in the field.

The genes identified in a particular individual and the cancer risk profile may be listed in a report, so the patient may have this information. This report may also be detailed enough to provide necessary information to the treating physician.

In an embodiment, the gene alteration and gene expression may be quantified. That is, by comparison of the gene expression in a cancer patient or group of cancer patients, with normal gene expression, a ranking of the dysfunction of the gene can be correlated with cancer severity. By repeating this analysis on several genes encoding comorbidities associated with cancers, a database of genes and their alterations can be created. This database may be a listing of relevant genes encoding comorbidities associated with cancers that can be used for predictive outcomes of cancer patients, and to develop therapeutic interventions based on gene alteration in a comorbidity gene.

In one embodiment, we selected the following set of genes from the list of high blood pressure genes: SCNN1B WNK1 WNK4 KCNJ5 CYP11B1 CYP11B2 PDE3A PRKG1 GUCY1A2. We then compared patients with stage 2 and lower cancer to stage 3 and higher cancer for difference in gene alterations, and gene expression. These genes were picked from the list of high blood pressure genes. The gene names comply with the HUGO gene nomenclature committee guidelines.

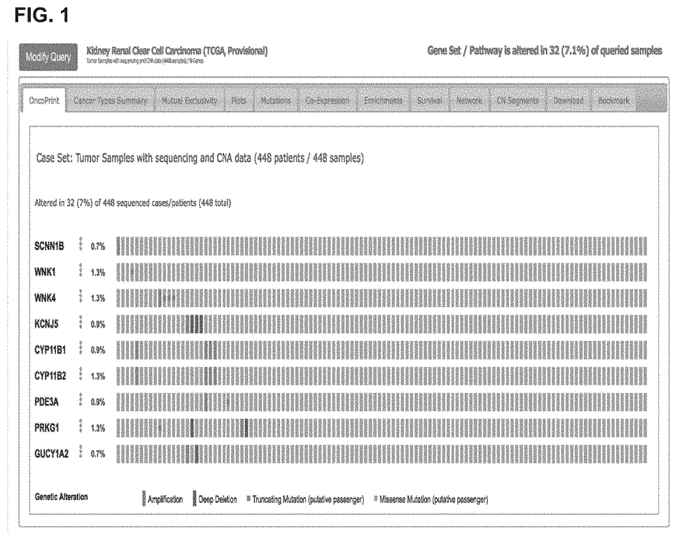

Analysis of Mutations. In accordance with one embodiment, we used an online resource to explore the significance of these genes. For example, we used the cBioPortal for Cancer Genomics (16) to identify subjects with renal cell carcinoma (clear cell type) in the TCGA (The Cancer Genome Atlas) catalog. (15) The TCGA is a project funded by the US government and is a catalogue of genetic mutations responsible for cancer using genome sequencing and bioinformatics. TCGA is a well-known project in cancer research that collects and analyzes high-quality tumor samples and makes the related data available to researchers. At the TCGA data portal, researchers can search, download, and analyze data from approximately 30 different tumor types. We identified a provisional data set comprising of 538 samples. We queried the TCGA website for the alterations in the genes noted above. We identified alterations in the genes shown in FIG. 1.

FIG. 1 shows gene mutations identified in a set of 448 patients in the TCGA database. The hypertension genes listed were altered in 32 (7%) of the 448 subjects. Specific mutations are shown in the gene maps of FIG. 1, and include amplification of certain segments, deep deletions, truncating mutations, and missense mutations.

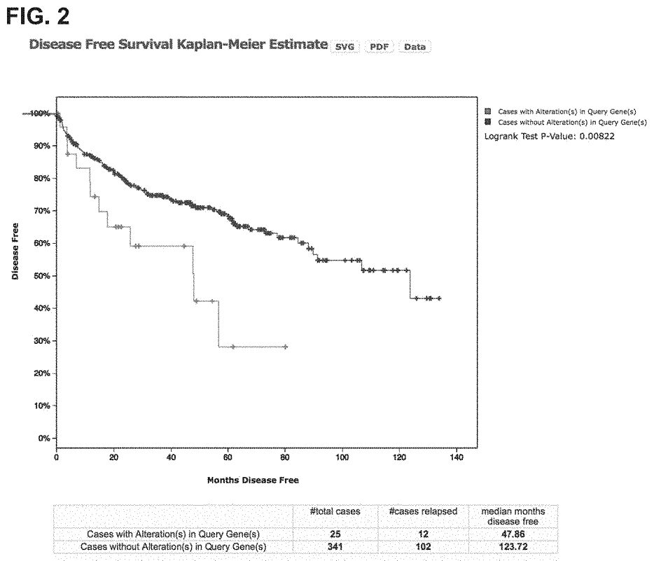

We also noted that the cancer specific survival was significantly worse for subjects with alterations in the said genes. (FIG. 2) The statistics depicted in FIG. 2 are a Kaplan-Meier survival plot wherein the cases with alterations had a significantly worse survival compared to cases without alterations. This was statistically significant using a Logrank test, with a p-value of 0.00822. This indicates that the patients with the alterations in the queried genes had worse survival which is not due to a chance or a flip of a coin, but due to an underlying phenomenon.

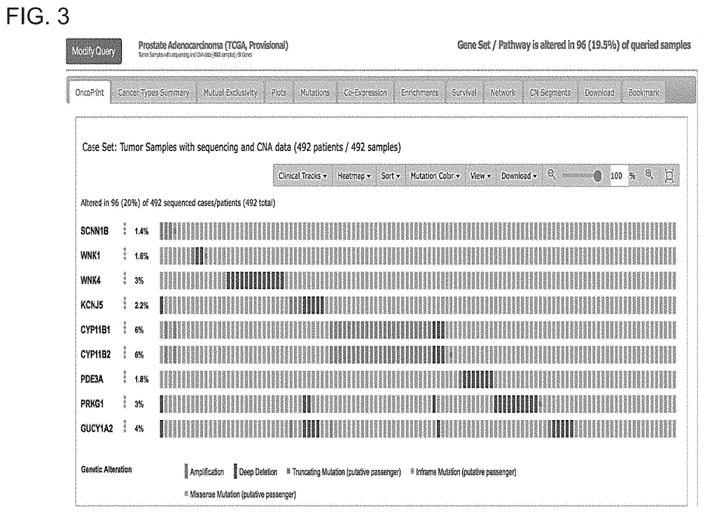

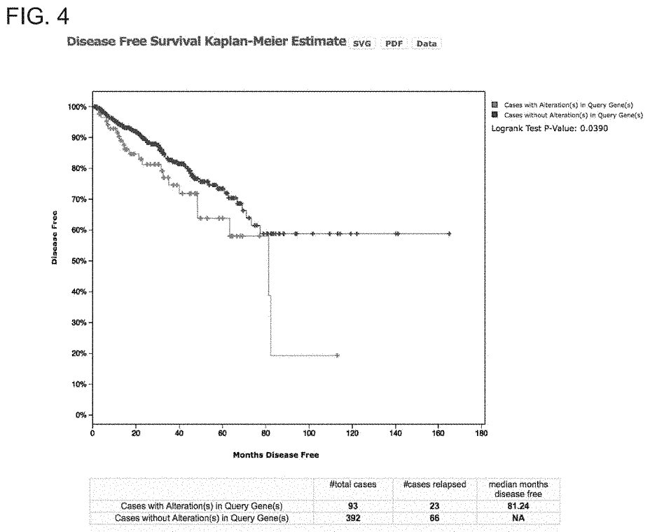

This same method was used to analyze gene alterations in prostate adenocarcinoma comorbidities (FIG. 3). We again used cBioPortal to identify a set of mutations, and the TCGA database to identify a set of 492 subjects having prostate adenocarcinoma and one or more of the listed comorbidity genes. We found that 96 subjects (20%) had gene mutations shown in the gene maps in FIG. 3. As with the renal cell carcinoma example, there was a significantly worse disease outcome in patients with the gene mutations, as shown in FIG. 4. The logrank test revealed a p-value of 0.039 again implying this was a significant difference between groups not related to chance but due an underlying phenomenon.

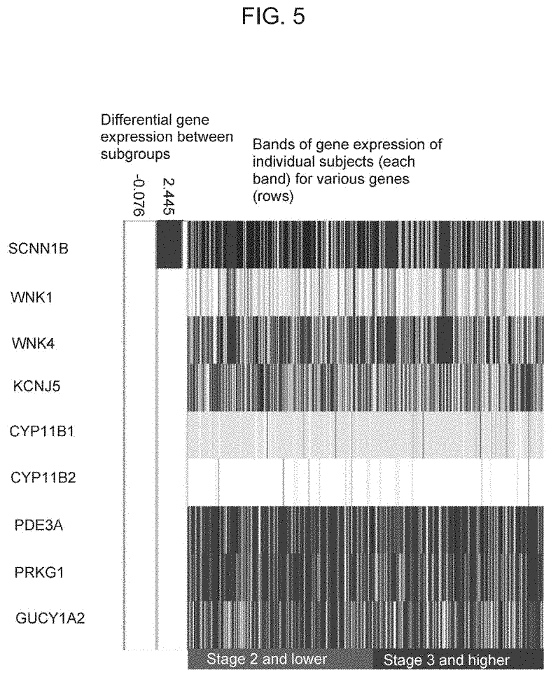

In another embodiment, we used the UCSC Xena tool (18) to explore the significance of these genes. We identified a data set of 538 samples with renal cell carcinoma (clear cell type) (ccRCC) in the TCGA. We queried the Xena tool to identify mutations in the said genes. We then checked them for a statistically significant difference between patients with low (stage pT2 and lower) and high (stage pT3 and higher) stage renal cell carcinoma (clear cell type). (FIG. 5) In FIG. 5, mutations towards the right of each row are associated with higher stage tumors and worse clinical outcomes. There is a more than 2-fold difference between the group stage 2 and lower compared to group stage 3 and higher for the expression of gene SCNN1 B. In other words, between patients with kidney cancer of clear cell type stage 2 or lower and patients with kidney cancer of clear cell type stage 3 and higher, who are statistically significant in terms of cancer specific survival rates, there is a significant difference in the expression of gene SCNN1B. Identifying this gene will help identify patients with this mutation through a diagnostic test, help in counseling patients so they can plan for the outcome of treatment of their cancer, and administer treatment to block, or alter the effect of this gene on the cancer progression.

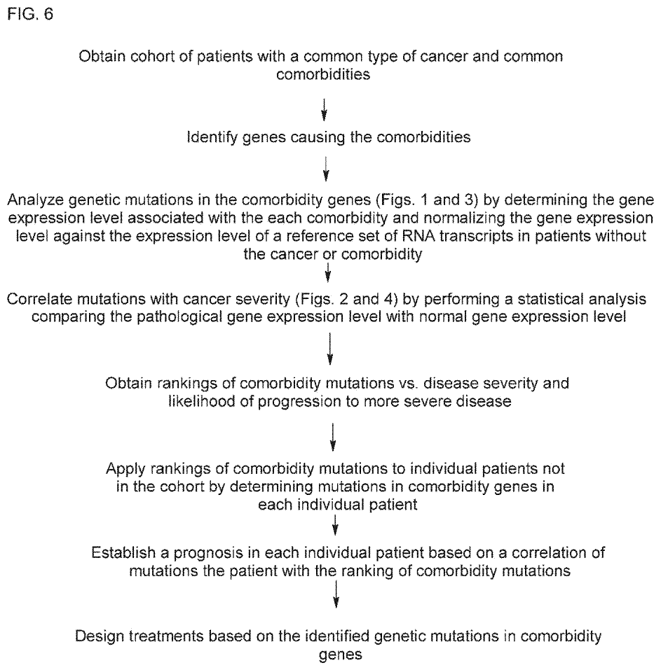

An embodiment of this method is shown as a flowchart in FIG. 6. A cohort of patents with a common type of cancer and common comorbidities (for example, hypertension or anemia) are identified. Candidate genes causing the comorbidity are identified. The genes are analyzed for genetic mutations, alterations, or differential gene expression. The mutations or alterations are correlated with markers of cancer progression, diagnosis, and prognosis. Rankings of mutations to various disease markers are thereby obtained. In an embodiment, gene expression levels are determined by normalizing the comorbidity gene expression level against the expression level of a reference set of RNA transcripts in patients without the cancer or comorbidity and performing a statistical analysis comparing the pathological gene expression level with normal gene expression level. “Normalization” of gene expression is the calculation of gene expression values to make it comparable in between different experiments. Several methods are used, few among them include housekeeping method, total RNA globalization method, centralization method, MAD method, and percentile normalization method among others. (19)

Once the gene expressions are normalized, the mutations or abnormalities in gene expression can be correlated with cancer severity (FIGS. 2 and 4) by performing a statistical analysis comparing the pathological gene expression level with normal gene expression level. In an embodiment, this statistical analysis quantifies gene alteration and gene expression as compared to normal gene expression. Rankings can be obtained of comorbidity mutations vs. disease severity and likelihood of progression to more severe disease. Thus, the genes as identified herein and the gene expression of those genes may be used to grade cancer outcomes for the cancer. This can be used as a predictive method of cancer survival.

In an embodiment, the gene alterations and mutations discussed above may directly impact oncogenes, that is, a mutated form of a gene involved in normal cell metabolism or growth, wherein the mutation causes uncontrolled cellular division or loss of cellular differentiation that is characteristic of tumors. Using genetic techniques, the interaction of altered comorbidity genes and oncogenes can be assessed. This analysis may be useful in elucidating mechanisms of action of altered comorbidity genes.

The rankings in FIG. 6 can be applied to other individual patients, not in the cohort, by determining mutations in genes related to comorbidities in the other individual patients. The mutations are used to estimate a prognosis for the individual patients. The mutations can also be used to plan treatments in the patients that intervene in the comorbidity pathology.

Clinical Applications. In an exemplary clinical scenario, a patient with renal cell carcinoma and hypertension comorbidity is evaluated for the risk of tumor progression. Having identified the genes associated with worse prognosis in as described above for RCC with hypertension comorbidity, a reverse transcriptase polymerase chain reaction (RT-PCR) platform can be used to identify the gene transcripts of the high blood pressure genes in the patient. These genes may also be combined into a microarray as known in the field, to facilitate assessment of the patient sample for the gene alterations or gene expressions of interest. Some other techniques known in the field to identify the gene alterations include whole exome sequencing, and other gene sequencing technologies. These techniques of identifying the set of gene alterations, or gene expression in a patient are prior knowledge, and can be used effectively to identify the gene alterations or gene expression in any given patient. The test may be performed on a biopsy of cancer tissue but could also be performed on organ(s) harboring the cancer, blood, or other body fluids, circulating tumor cells, or stored tissue from the patient.

The test could be performed serially in time to assess the changes in the genes of interest over time. The test sample if necessary is collected and stored in tubes that stabilize and prevent degradation of nucleotides or proteins of interest. The gene expressions are normalized against the expression levels of all RNA transcripts or their expression products in the tumor being evaluated, or a reference set of RNA transcripts or their products. If the gene alterations or the gene transcripts identified are among the genes associated with high risk for progression as identified above, the patient can then be appropriately counseled on the appropriate treatment.

For example, a treatment could directly address the comorbidity, or could be agents that block the mutated gene(s) in that patient, or agents that block the products of the gene(s). For example, if the comorbidity is hypertension, the treatment may be blood pressure lowering drugs. Further, serial measurement of the alterations in the gene or gene products could provide information related to the progression of the disease. Additionally, potential treatments include blood pressure lowering agents and agents that block the by products of these genes, which can play a role in halting, reversing, or limiting the progression of the cancer.

Similar methods can be used to identify individuals potentially at higher risk of harboring high risk RCC. Additionally, potential treatments include blood pressure lowering agents and agents that block the byproducts of these genes, which can play a role in halting, reversing, or limiting the progression of the cancer.

This method is not limited to clear cell type renal cell carcinoma or prostate cancer and can be extrapolated to other tumor types. Similarly, this method is not limited to genes encoding hypertension as a comorbidity. The method is not limited to two groups of stage 2 and lower compared to stage 3 and higher. The comparison groups may include stage 1 to stage 2 and higher; stage 3 and lower compared to stage 4 and higher; or between any tumor classification types or between any tumor groups comparing lower to higher risk groups, as long as there is a statistically significant difference between the groups can be demonstrated. The difference in the genes can be used to identify individuals potentially at higher risk of harboring high risk cancer and more likely to have a worse outcome.

A number of exemplary genes are shown in Tables 1 and 2 attached to this disclosure. These tables provide several hundred genes associated with comorbidities that are linked to various cancers.

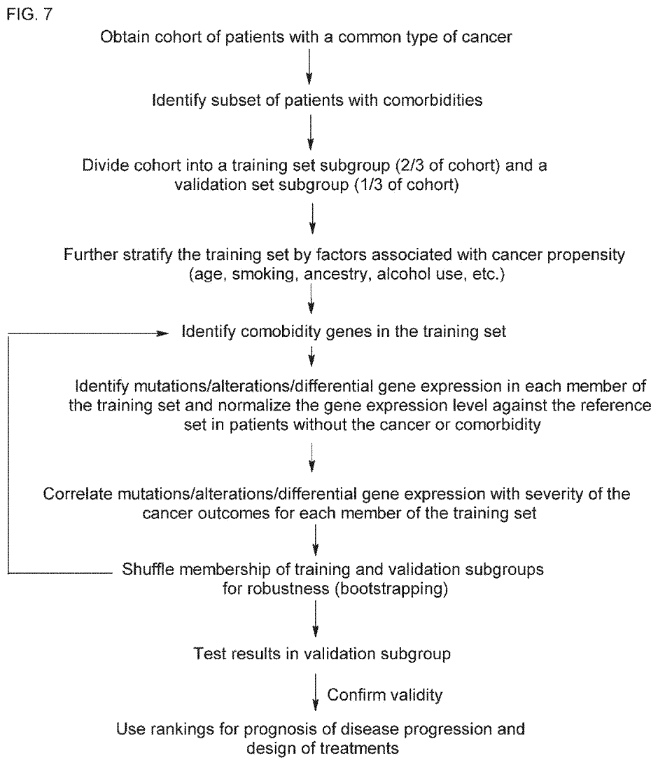

Use of a Training Set. In another embodiment, artificial intelligence methods can be used to identify genetic mutations in comorbidities in cancer patients. Results can be refined by bootstrapping. The methods can be used diagnostically to stage cancers, and to prescribe targeted treatment for cancers in which comorbidities are a cause or a cofactor. This is illustrated in the flow chart in FIG. 7.

In an embodiment, a cohort of patients (FIG. 7) with the same type of cancer is selected. The cohort is analyzed to identify comorbidities associated with the cancer. The cohort is divided into a training set (for example, ⅔rd of the cohort), and a validation set (for example, ⅓rd of the cohort). This division of the cohort may be done by randomly assigning patients into each of the sets or based on various factors associated with cancer propensity. For example, the two groups could have different tumor stages, age (e.g.: >70 years versus <=70 years), smokers vs non-smokers, alcoholics versus non-alcoholics, gender (male versus female), hormonal status (normal versus abnormal hormone levels or response), tumor versus controls, metastatic versus non-metastatic disease, or any other parameters to assess gene alterations and their differential expression. More than two groups could be created.

In the training set, comorbidity genes are identified and statistically different DNA mutations in the comorbidity genes are identified, for example, from mutations causing methylation, differential gene expression, RNA and protein expression of genes in the training set and normal gene expression. The genes of interest can also be modified. For example, if a patient has a certain altered gene, we could look for that gene in this model. Other methods of identifying genes of interest include any other well-known statistical methods in the field. One such method is to identify (for example) the top 5, 10, or 20 altered genes by this method.

The genetic mutations, alterations, and differential expression in the comorbidity genes are correlated with cancer severity in the training set by determining the gene expression level associated with each comorbidity and normalizing the gene expression level against the expression level of a reference set of RNA transcripts in patients without the cancer or comorbidity. The correlation may be used to obtain a ranking of mutations. In the validation set, we can confirm if the results obtained from the analysis of the training set to identify statistically different DNA mutations, methylation, differential gene expression, RNA and protein expression of genes lead to statistically significant differences in cancer severity in the validation set.

For example, if ten altered genes are found to be leading indicators of a particular cancer in the training set, the analysis will seek to confirm if patients in the validation set have statistically similar gene alterations. The statistical analysis could be any method by which a person with knowledge in the field would deem relevant for distinguishing the plurality of groups to have significantly different outcomes.

In an embodiment, a bootstrapping random resampling method may be employed to refine the results. (20) In this technique, the training and validation sets are shuffled, so that one or more members of the sets are swapped. The above training set embodiment may be repeated one or more times on different permutations of the training set and the validation set. That is, a new set of training and validation assignments may be made in the cohort and the statistical analysis is repeated. This process can be automated using a computer. This reassignment can be repeated many times with different combinations of training and validation set members, and the correlation to cancer severity can be determined. The reassignment can be repeated with as many permutations of membership in the training and validation sets in the cohort are possible. Repetitions of dozens, hundreds, or thousands of analyses can be performed with a bootstrapping method of shuffling membership of the training and validation sets and repeating the analysis. The statistical analysis is then repeated by identifying genetic mutations, alterations, and differential expression in the comorbidity genes and the cancer related outcomes.

For example, a cohort of 60 cancer patients may be studied, in which 30 have hypertension and 30 do not have hypertension. The cohort is then randomly divided into a training set (40 patients) and a validation set (20 patients). In this instance, 20 patients with hypertension and 20 patients without hypertension may be in the training set while 10 remaining patients with hypertension and without hypertension may be in the validation set. In the repeat analysis, one or more patients from the training set is replaced (i.e., swapped) with an equal number of patients in the validation set (this is referred to in FIG. 7 as shuffling the membership of the training and validation sets). This replacement can be repeated many times. A computer algorithm can do this any number of times as long as each experiment is not repeated with the same set of patients. This is one method to build robustness to a statistical finding.

Ultimately, the results are tested in the validation group to confirm the validity. If the rankings are valid, they can be used diagnostically to stage cancer severity, for prognosis to estimate the likelihood of progression to more severe disease, and for treatment, to design therapeutic interventions in particular that affect comorbidities.

There are several ways to perform statistical methods, and any such common knowledge methods can be used to identify the genes of interest. Once a set of altered genes is identified, the comorbidity, gene, or gene products can then be evaluated for potential therapeutic targets, thereby achieving either a cure, or a delay in progression of cancer.

In an embodiment, angiotensin receptor blockers, a class of blood pressure medications, may be used to reduce the incidence of kidney cancer, and/or progression of kidney cancer in kidney cancer patients having a hypertension comorbidity. (21) In an embodiment, the thiazolidinediones which represent a class of transcription-modulating drugs that exert effects on blood pressure, carbohydrate and lipid metabolism, and vascular growth and function can be used to treat the underlying comorbidity such as diabetes type-2. Resources such as “reactome” (21) and “KEGG PATHWAY” (22) can provide tools to explore drug targets, the method of which is within the realm of one skilled in the art.

In another embodiment, the gene alterations identified by statistical analyses may be ranked based on their close association with known cancer genes. The single-gene analysis method is a conventional statistical analysis of the gene expression data that examines one gene at a time. The method determines the differentially expressed (DE) levels of the gene in different phenotypes and then makes adjustments to the levels for multiple gene testing. This method, however, possesses several limitations: high-ranking genes may score highly simply by chance, given the large number of hypotheses involved; significant genes may show distressingly little overlap among different studies of the same biological system; and analysis may miss important effects of sets of genes in pathways. Because of the limitations of single-gene analysis, researchers have increasingly turned to the development of gene set analysis methods, which consider a set of genes as a whole and determine its correlation with disease phenotypes based on the differing levels of the genes' expression. Different gene set analysis methods, which either find gene sets that were previously unknown or select gene sets in a known collection (such as known pathways), have been proposed for genomic data analysis.

Gene Set Enrichment Analysis (GSEA) uses overrepresentation analysis to determine if given sets of genes are DE in different disease phenotypes and has been widely adopted to analyze data in biological experiments. The goal of GSEA is to determine if members of a gene set tend to occur toward the top of the gene list because of the genes' correlation with the phenotypic class distinction. The given gene set can be a set of genes in a pathway, a set of genes in a gene ontology category, or any user-defined set.

The complex procedure of finding pathway abnormalities in cancer could have many steps involved, such as information extraction from biological data, simulation verification, biological experimental testing, and clinical trials. Among these steps, analysis based on biological data to determine the relationship of pathways (and the gene sets therein) to a certain cancer is one of the most important steps. For example, the relationship of signaling pathways of genes involved in the comorbidities, and a certain cancer type in which Fisher's exact test may be used to identify the related pathways based on the significance level of DE genes. Another method is to use the supervised analysis of messenger RNA microarray data from human tumors.

The statistical analysis may also include multivariate analysis, with one more of the patient baseline characteristics data such as age, comorbidity, gender, race, gene alteration data, gene expression data being included in such multivariate analysis. Other common methods of statistical analysis known in the art may also be used. The links for some of the software available in the field are “Bioconductor” (23) and “TCGA Biolinks”. (24)

Accordingly, we have provided a method to identify genes associated with medical comorbidities predicting worse cancer related outcomes. The method further provides for predicting cancer related risk of progression to an individual patient. The method further provides for treating cancer by identifying comorbidities in cancer patients, identifying genes associated with the comorbidities, and interrupting the comorbidities with therapeutic interventions directed at the specific genes identified with the comorbidity.

### Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed, J. Wiley & Sons (New York N. Y. 1994), and, Advanced Organic Chemistry Reactions, Mechanisms and Structure 4th ed., John Wiley & Sons (New York, N.Y. 1992), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “microarray” refers to an ordered arrangement of hybridizable array elements, preferably polynucleotide probes, on a substrate.

The term “polynucleotide”, when used in singular or plural, generally refers to any polyribonucleotide or polydeoxy-ribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical regions often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein.

The terms “differentially expressed gene”, “differential gene expression” and their synonyms, which are used interchangeably, refer to a gene whose expression is activated to a higher or lower level in a subject suffering from a disease, specifically cancer, such as kidney cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering form a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and disease subjects, or in various stages of disease development in a diseased subject.

The phrase “gene amplification” refers to a process by which multiple copies of a gene or gene fragment are formed in a particular cell or cell line. The duplicated region (a stretch of amplified DNA) is often referred to as “amplicon”. Usually, the amount of the messenger RNA (mRNA) produced, i.e., the level of gene expression, also increases in the proportion of the number of copies made of the particular gene expressed.

The term “diagnosis” is used herein to refer to the identification of a molecular or pathological state, disease or condition, such as the identification of a molecular subtype of head and neck cancer, colon cancer, or other type of cancer.

The term “prognosis” is used herein to refer to the prediction of the likelihood of cancer-attributable death or progression, including recurrence, metastatic spread, and drug resistance, of a neoplastic disease, such as breast cancer.

The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “tumor” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, kidney cancer, prostate cancer, bladder cancer, breast cancer, lung cancer, colon cancer, hepatocellular cancer, gastric cancer, pancreatic cancer, cervical cancer, ovarian cancer, liver cancer, cancer of the urinary tract, thyroid cancer, melanoma and brain. Any other terms used in this application must be used in the context of use and interpretation as used by one skilled in the art.

The practice of the present invention will employ, unless otherwise indicated, conventional techniques of molecular biology (including recombinant techniques), microbiology, cell biology, and biochemistry, which are within the skill of the art. “Molecular Cloning” A Laboratory Manual”, 2nd edition (Sambrook et al., 1989); Parker & Barnes, mRNA: Detection by In Situ and Northern Hybridization. (25)

### REFERENCES

2. Uzunlulu, M.; Telci Caklili, O.; Oguz, A., Association between Metabolic Syndrome and Cancer. Annals of Nutrition and Metabolism 2016, 68, 173-179, DOI: 10.1159/000443743.
3. Frank, I.; Blute, M. L.; Cheville, J. C.; Lohse, C. M.; Weaver, A. L.; Zincke, H., Solid renal tumors: an analysis of pathological features related to tumor size. J Urol 2003, 170, 2217-20, DOI: 10.1097/01.ju.0000095475.12515.5e.
4. Sahni, V. A.; Silverman, S. G., Imaging Management of Incidentally Detected Small Renal Masses. Semin intervent Radiol 2014, 31, 009-019, DOI: 10.1055/s-0033-1363838.
5. Cancer Staging. https://www.cancer.gov/about-cancer/diagnosis-staging/staging (Accessed: 9/20/2021).
6. Melamed, R. D.; Emmett, K. J.; Madubata, C.; Rzhetsky, A.; Rabadan, R., Genetic similarity between cancers and comorbid Mendelian diseases identifies candidate driver genes. Nature Communications 2015, 6, 7033, DOI: 10.1038/ncomms8033.
7. OMIM®, Online Mendelian Inheritance in Man®. https://omim.org/ (Accessed: 9/22/2021).
8. Li, Y. H.; Zhang, G. G.; Wang, N., Systematic Characterization and Prediction of Human Hypertension Genes. Hypertension 2017, 69, 349-355, DOI: 10.1161/hypertensionaha.116.08573.
9. Feinstein, A. R., The pre-therapeutic classification of co-morbidity in chronic disease. Journal of Chronic Diseases 1970, 23, 455-468, DOI: https://doi.org/10.1016/0021-9681(70)90054-8.
10. Wang, X.; Gotoh, O., Accurate molecular classification of cancer using simple rules. BMC Med Genomics 2009, 2, 64, DOI: 10.1186/1755-8794-2-64.
11. Golub, T. R.; Slonim, D. K.; Tamayo, P.; Huard, C.; Gaasenbeek, M.; Mesirov, J. P.; Cotler, H.; Loh, M. L.; Downing, J. R.; Caligiuri, M. A.; Bloomfield, C. D.; Lander, E. S., Molecular classification of cancer: class discovery and class prediction by gene expression monitoring. Science 1999, 286, 531-7, DOI: 10.1126/science.286.5439.531.
12. Services, C. f. D. a. M. ICD-10 CM. https://www.cms.gov/medicare/icd-10/2021-icd-10-cm (Accessed:
13. McLaughlin, J. K.; Chow, W. H.; Mandel, J. S.; Mellemgaard, A.; McCredie, M.; Lindblad, P.; Schlehofer, B.; Pommer, W.; Niwa, S.; Adami, H.-O., International renal-cell cancer study. VIII. Role of diuretics, other anti-hypertensive medications and hypertension. International Journal of Cancer 1995, 63, 216-221, DOI: https://doi.org/10.1002/ijc.2910630212.
14. Human Phenotype Ontology (HPO). https://hpo.jax.org/app/ (Accessed: Sep. 23, 2021).
15. Cancer Genome Atlas (TCGA). https://portal.gdc.cancer.gov/ (Accessed: 9/23/2021).
16. cBioPortal for Cancer Genomics https://www.cbioportal.org/ (Accessed: Sep. 23, 2021).
17. Dai, H.-J.; Wu, J. C.-Y.; Tsai, R. T.-H.; Pan, W.-H.; Hsu, W.-L., T-HOD: a literature-based candidate gene database for hypertension, obesity and diabetes. Database 2013, 2013, DOI: 10.1093/database/bas061.
18. UCSC Xena. https://xena.ucsc.edu/welcome-to-ucsc-xena/ (Accessed: Sep. 23, 2021).
19. Fundel, K.; Haag, J.; Gebhard, P. M.; Zimmer, R.; Aigner, T., Normalization strategies for mRNA expression data in cartilage research. Osteoarthritis Cartilage 2008, 16, 947-55, DOI: 10.1016/j.joca.2007.12.007.
20. Wu, C. F. J., Jackknife, Bootstrap and Other Resampling Methods in Regression Analysis. The Annals of Statistics 1986, 14, 1261-1295, 35, DOI: 10.1214/aos/1176350142.
21. reactome.org. https://reactome.org/ (Accessed: Oct. 6, 2021).
22. KEGG PATHWAY Database. https://www.genome.jp/kegg/pathway.html (Accessed: Oct. 6, 2021).
23. Bioconductor. https://bioconductor.org/ (Accessed: Oct. 6, 2021).
24. TCGAbiolinks: Clinical data. https://www.bioconductor.org/packages/devel/bioc/vignettes/TCGAbiolinks/inst/doc/clini cal.html (Accessed: Oct. 6, 2021).
25. Parker, R. M. C.; Barnes, N. M., mRNA: Detection by In Situ and Northern Hybridization. In Receptor Binding Techniques, Keen, M., Ed. Springer New York: Totowa, N.J., 1999; pp 247-283. 10.1385/0-89603-530-1:247

TABLE 1

| Gene | Associated diseases |
|---|---|
| MKKS | MCKUSICK-KAUFMAN SYNDROME |
| (8195) | (ORPHA:2473), BARDET-BIEDL SYNDROME 6 |
| | (OMIM:605231), BARDET-BIEDL |
| | SYNDROME (ORPHA:110), BARDET-BIEDL |
| | SYNDROME 1 (OMIM:209900), MCKUSICK- |
| | KAUFMAN SYNDROME (OMIM:236700) |
| TET2 | MYELODYSPLASTIC SYNDROME |
| (54790) | (OMIM:614286), POLYCYTHEMIA VERA |
| | (ORPHA:729), ESSENTIAL |
| | THROMBOCYTHEMIA (ORPHA:3318) |
| IL12A | PRIMARY BILIARY CHOLANGITIS |
| (3592) | (ORPHA:186), BEHÃ‡ET DISEASE (ORPHA:117) |
| HGD (3081) | ALKAPTONURIA (ORPHA:56), |
| | ALKAPTONURIA (OMIM:203500) |
| IL12B | IMMUNODEFICIENCY 29 (OMIM:614890), |
| (3593) | TAKAYASU ARTERITIS (ORPHA:3287) |
| TMEM67 | MECKEL SYNDROME (ORPHA:564), |
| (91147) | JOUBERT SYNDROME WITH HEPATIC |
| | DEFECT (ORPHA:1454), NEPHRONOPHTHISIS |
| | 11 (OMIM:613550), JOUBERT |
| | SYNDROME (ORPHA:475), JOUBERT |
| | SYNDROME 6 (OMIM:610688), COACH |
| | SYNDROME (OMIM:216360), MECKEL |
| | SYNDROME, TYPE 3 (OMIM:607361) |
| IL12RB1 | PRIMARY BILIARY CHOLANGITIS |
| (3594) | (ORPHA:186), IMMUNODEFICIENCY 30 |
| | (OMIM:614891) |
| POU6F2 | NEPHROBLASTOMA (ORPHA:654) |
| (11281) | |
| PDE3A | BRACHYDACTYLY-ARTERIAL |
| (5139) | HYPERTENSION SYND . . . (ORPHA:1276), |
| | HYPERTENSION AND BRACHYDACTYLY |
| | SYNDROME (OMIM:112410) |
| MYH9 | DEAFNESS, AUTOSOMAL DOMINANT |
| (4627) | NONSYNDROMI . . . (OMIM:603622), |
| | SEBASTIAN SYNDROME (OMIM:605249), |
| | MACROTHROMBOCYTOPENIA AND |
| | PROGRESSIVE SE . . . (OMIM:600208), |
| | MAY-HEGGLIN ANOMALY (OMIM:155100), |
| | FECHTNER SYNDROME (OMIM:153640), |
| | EPSTEIN SYNDROME (OMIM:153650) |
| MYH11 | MEGACYSTIS-MICROCOLON-INTESTINAL |
| (4629) | HYPOPER . . . (ORPHA:2241), FAMILIAL |
| | AORTIC DISSECTION (ORPHA:229), AORTIC |
| | ANEURYSM, FAMILIAL THORACIC 4 |
| | (OMIM:132900), FAMILIAL THORACIC |
| | AORTIC ANEURYSM AND AO . . . |
| | (ORPHA:91387) |
| EOGT | ADAMS-OLIVER SYNDROME (ORPHA:974), |
| (285203) | ADAMS-OLIVER SYNDROME 4 |
| | (OMIM:615297) |
| ERCC4 | FANCONI ANEMIA (ORPHA:84), XERODERMA |
| (2072) | PIGMENTOSUM (ORPHA:910), |
| | XERODERMA PIGMENTOSUM-COCKAYNE |
| | SYNDROME . . . (ORPHA:220295), |
| | XERODERMA PIGMENTOSUM, |
| | COMPLEMENTATION G . . . (OMIM:278760), |
| | TRACHEOESOPHAGEAL FISTULA WITH |
| | OR WITHOU . . . (OMIM:189960), XFE |
| | PROGEROID SYNDROME (OMIM:610965), |
| | FANCONI ANEMIA, COMPLEMENTATION |
| | GROUP Q (OMIM:615272) |
| PRTN3 | GRANULOMATOSIS WITH POLYANGIITIS |
| (5657) | (ORPHA:900) |
| DIS3L2 | NEPHROBLASTOMA (ORPHA:654), |
| (129563) | PERLMAN SYNDROME (OMIM:267000) |
| ERCC6 | UV-SENSITIVE SYNDROME 1 (OMIM:600630), |
| (2074) | COCKAYNE SYNDROME, TYPE B |
| | (OMIM:133540), COFS SYNDROME |
| | (ORPHA:1466), DE SANCTIS-CACCHIONE |
| | SYNDROME (OMIM:278800), |
| | CEREBROOCULOFACIOSKELETAL SYNDROME 1 |
| | (OMIM:214150) |
| ZMPSTE24 | MANDIBULOACRAL DYSPLASIA WITH |
| (10269) | TYPE B LIP . . . (ORPHA:90154), |
| | HUTCHINSON-GILFORD PROGERIA |
| | SYNDROME (ORPHA:740), |
| | MANDIBULOACRAL DYSPLASIA WITH |
| | TYPE B LIP . . . (OMIM:608612), RESTRICTIVE |
| | DERMOPATHY, LETHAL (OMIM:275210) |
| MYLK | AORTIC ANEURYSM, FAMILIAL THORACIC |
| (4638) | 7 (OMIM:613780), FAMILIAL THORACIC |
| | AORTIC ANEURYSM AND AO . . . (ORPHA:91387) |
| SPIB (6689) | PRIMARY BILIARY CHOLANGITIS (ORPHA:186) |
| HLA-B | BEHÃ‡ET DISEASE (ORPHA:117), |
| (3106) | TAKAYASU ARTERITIS (ORPHA:3287), |
| | STEVENS-JOHNSON SYNDROME (ORPHA:36426) |
| TRAF3IP1 | SEN IOR-LOKEN SYNDROME 9 (OMIM:616629), |
| (26146) | SENIOR-LOKEN SYNDROME |
| | (ORPHA:3156) |
| TMEM237 | JOUBERT SYNDROME WITH OCULORENAL |
| (65062) | DEFECT (ORPHA:2318), JOUBERT |
| | SYNDROME (ORPHA:475), JOUBERT |
| | SYNDROME WITH RENAL DEFECT |
| | (ORPHA:220497), JOUBERT SYNDROME |
| | 14 (OMIM:614424) |
| LEMD3 | MELORHEOSTOSIS, ISOLATED |
| (23592) | (OMIM:155950), BUSCHKE-OLLENDORFF |
| | SYNDROME (OMIM:166700), BUSCHKE- |
| | OLLENDORFF SYNDROME (ORPHA:1306), |
| | MELORHEOSTOSIS WITH OSTEOPOIKILOSIS |
| | (ORPHA:1879), ISOLATED |
| | OSTEOPOIKILOSIS (ORPHA:166119), |
| | 12Q14 MICRODELETION SYNDROME |
| | (ORPHA:94063) |
| AVPR2 | DIABETES INSIPIDUS, NEPHROGENIC, |
| (554) | X-LINKE . . . (OMIM:304800), NEPHROGENIC |
| | SYNDROME OF INAPPROPRIATE AN . . . |
| | (OMIM:300539), NEPHROGENIC DIABETES |
| | INSIPIDUS (ORPHA:223) |
| HLA-DPB1 | GRANULOMATOSIS WITH POLYANGIITIS |
| (3115) | (ORPHA:900) |
| ENPP1 | HYPOPHOSPHATEMIC RICKETS, AUTOSOMAL |
| (5167) | RECE . . . (OMIM:613312), |
| | PSEUDOXANTHOMA ELASTICUM |
| | (ORPHA:758), ARTERIAL CALCIFICATION, |
| | GENERALIZED, OF . . . (OMIM:208000), |
| | COLE DISEASE (OMIM:615522) |
| CYP11B1 | GLUCOCORTICOID-REMEDIABLE |
| (1584) | ALDOSTERONISM (OMIM:103900), FAMILIAL |
| | HYPERALDOSTERONISM TYPE I |
| | (ORPHA:403), CONGENITAL ADRENAL |
| | HYPERPLASIA DUE TO 11 . . . (ORPHA:90795), |
| | ADRENAL HYPERPLASIA, |
| | CONGENITAL, DUE TO . . . (OMIM:202010) |
| IFT172 | RETINITIS PIGMENTOSA (ORPHA:791), |
| (26160) | SHORT-RIB THORACIC DYSPLASIA 10 |
| | WITH OR . . . (OMIM:615630), JEUNE |
| | SYNDROME (ORPHA:474), RETINITIS |
| | PIGMENTOSA 71 (OMIM:616394), |
| | BARDET-BIEDL SYNDROME (ORPHA:110) |
| CYP11B2 | FAMILIAL HYPERALDOSTERONISM |
| (1585) | TYPE I (ORPHA:403), CORTICOSTERONE |
| | METHYLOXIDASE TYPE I DEFI . . . |
| | (OMIM:203400), CORTICOSTERONE |
| | METHYLOXIDASE TYPE II DEF . . . |
| | (OMIM:610600) |
| CYP17A1 | CONGENITAL ADRENAL HYPERPLASIA |
| (1586) | DUE TO 17 . . . (ORPHA:90793), 46, XY |
| | DISORDER OF SEX DEVELOPMENT DUE |
| | TO . . . (ORPHA:90796), ADRENAL |
| | HYPERPLASIA, CONGENITAL, DUE |
| | TO . . . (OMIM:202110) |
| HLA-DRB1 | DIFFUSE CUTANEOUS SYSTEMIC |
| (3123) | SCLEROSIS (ORPHA:220393), FOLLICULAR |
| | LYMPHOMA (ORPHA:545), SYSTEMIC-ONSET |
| | JUVENILE IDIOPATHIC ARTHR . . . |
| | (ORPHA:85414), NARCOLEPSY WITHOUT |
| | CATAPLEXY (ORPHA:83465), LIMITED |
| | CUTANEOUS SYSTEMIC SCLEROSIS |
| | (ORPHA:220402), NARCOLEPSY- |
| | CATAPLEXY SYNDROME (ORPHA:2073), |
| | BULLOUS PEMPHIGOID (ORPHA:703) |
| CYP21A2 | ADRENAL HYPERPLASIA, CONGENITAL, |
| (1589) | DUE TO . . . (OMIM:201910) |
| SDCCAG8 | SENIOR-LOKEN SYNDROME 7 (OMIM:613615), |
| (10806) | BARDET-BIEDL SYNDROME |
| | (ORPHA:110), BARDET-BIEDL SYNDROME 1 |
| | (OMIM:209900), BARDET-BIEDL |
| | SYNDROME 16 (OMIM:615993), |
| | SENIOR-LOKEN SYNDROME (ORPHA:3156) |
| B2M (567) | VARIANT ABETA2M AMYLOIDOSIS |
| | (ORPHA:314652), AMYLOIDOSIS, FAMILIAL |
| | VISCERAL (OMIM:105200), |
| | HYPOPROTEINEMIA, HYPERCATABOLIC |
| | (OMIM:241600) |
| KIF1B | PHEOCHROMOCYTOMA (OMIM:171300), |
| (23095) | CHARCOT-MARIE-TOOTH DISEASE, |
| | AXONAL, TYP . . . (OMIM:118210) |
| CD2AP | FOCAL SEGMENTAL GLOMERULOSCLEROSIS |
| (23607) | 3, SU . . . (OMIM:607832) |
| ACTA2 (59) | MOYAMOYA DISEASE (ORPHA:2573), |
| | MOYAMOYA DISEASE 5 (OMIM:614042), |
| | AORTIC ANEURYSM, FAMILIAL THORACIC |
| | 6 (OMIM:611788), MULTISYSTEMIC |
| | SMOOTH MUSCLE DYSFUNCTION . . . |
| | (OMIM:613834), FAMILIAL THORACIC AORTIC |
| | ANEURYSM AND AO . . . (ORPHA:91387) |
| BBS1 (582) | BARDET-BIEDL SYNDROME (ORPHA:110), |
| | BARDET-BIEDL SYNDROME 1 |
| | (OMIM:209900) |
| BBS2 (583) | RETINITIS PIGMENTOSA (ORPHA:791), |
| | BARDET-BIEDL SYNDROME 2 |
| | (OMIM:615981), BARDET-BIEDL |
| | SYNDROME (ORPHA:110), RETINITIS |
| | PIGMENTOSA 74 (OMIM:616562) |
| GBE1 | POLYGLUCOSAN BODY DISEASE, |
| (2632) | ADULT FORM (OMIM:263570), ADULT |
| | POLYGLUCOSAN BODY DISEASE |
| | (ORPHA:206583), GLYCOGEN STORAGE |
| | DISEASE IV (OMIM:232500) |
| HMBS | PORPHYRIA, ACUTE INTERMITTENT |
| (3145) | (OMIM:176000), ACUTE INTERMITTENT |
| | PORPHYRIA (ORPHA:79276) |
| BBS4 (585) | BARDET-BIEDL SYNDROME 4 |
| | (OMIM:615982), BARDET-BIEDL SYNDROME |
| | (ORPHA:110), BARDET-BIEDL |
| | SYNDROME 1 (OMIM:209900) |
| PTPN22 | OLIGOARTICULAR JUVENILE ARTHRITIS |
| (26191) | (ORPHA:85410), GRANULOMATOSIS |
| | WITH POLYANGIITIS (ORPHA:900), |
| | JUVENILE RHEUMATOID FACTOR-NEGATIVE |
| | POLY . . . (ORPHA:85408), GIANT CELL |
| | ARTERITIS (ORPHA:397), VOGT-KOYANAGI- |
| | HARADA DISEASE (ORPHA:3437) |
| HPSE2 | OCHOA SYNDROME (ORPHA:2704), |
| (60495) | UROFACIAL SYNDROME (OMIM:236730) |
| IRF5 (3663) | DIFFUSE CUTANEOUS SYSTEMIC |
| | SCLEROSIS (ORPHA:220393), LIMITED |
| | CUTANEOUS SYSTEMIC SCLEROSIS |
| | (ORPHA:220402), PRIMARY BILIARY |
| | CHOLANGITIS (ORPHA:186) |
| EXT2 (2132) | EXOSTOSES, MULTIPLE, TYPE II |
| | (OMIM:133701), SEIZURES, SCOLIOSIS, AND |
| | MACROCEPHALY SY . . . (OMIM:616682), |
| | POTOCKI-SHAFFER SYNDROME |
| | (ORPHA:52022), MULTIPLE |
| | OSTEOCHONDROMAS (ORPHA:321) |
| KCTD1 | SCALP-EAR-NIPPLE SYNDROME |
| (284252) | (ORPHA:2036), SCALP-EAR-NIPPLE SYNDROME |
| | (OMIM:181270) |
| ACVRL1 | TELANGIECTASIA, HEREDITARY |
| (94) | HEMORRHAGIC, . . . (OMIM:600376), HEREDITARY |
| | HEMORRHAGIC TELANGIECTASIA (ORPHA:774) |
| RREB1 | 22Q11.2 DELETION SYNDROME (ORPHA:567) |
| (6239) | |
| GPR101 | PITUITARY ADENOMA, GROWTH |
| (83550) | HORMONE-SECRET . . . (OMIM:300943), |
| | ACROMEGALY (ORPHA:963) |
| GDF2 | TELANGIECTASIA, HEREDITARY |
| (2658) | HEMORRHAGIC, . . . (OMIM:615506), HEREDITARY |
| | HEMORRHAGIC TELANGIECTASIA (ORPHA:774) |
| WNK1 | HEREDITARY SENSORY AND AUTONOMIC |
| (65125) | NEUROPA . . . (ORPHA:970), |
| | PSEUDOHYPOALDOSTERONISM, TYPE IIC |
| | (OMIM:614492), NEUROPATHY, |
| | HEREDITARY SENSORY AND AUTON . . . |
| | (OMIM:201300) |
| NPHP4 | NEPHRONOPHTHISIS 4 (OMIM:606966), |
| (261734) | SENIOR-LOKEN SYNDROME 4 |
| | (OMIM:606996), SENIOR-LOKEN SYNDROME |
| | (ORPHA:3156) |
| ADA2 | SNEDDON SYNDROME (ORPHA:820), |
| (51816) | SNEDDON SYNDROME (OMIM:182410), |
| | POLYARTERITIS NODOSA, |
| | CHILDHOOD-ONSET (OMIM:615688) |
| F5 (2153) | BUDD-CHIARI SYNDROME (ORPHA:131), |
| | FACTOR V DEFICIENCY (OMIM:227400), |
| | THROMBOPHILIA DUE TO DEFICIENCY |
| | OF ACTIV . . . (OMIM:188055) |
| BBS9 | BARDET-BIEDL SYNDROME 9 (OMIM:615986), |
| (27241) | BARDET-BIEDL SYNDROME |
| | (ORPHA:110) |
| PTGIS | HYPERTENSION, ESSENTIAL (OMIM:145500) |
| (5740) | |
| ALX4 | PARIETAL FORAMINA 2 (OMIM:609597), |
| (60529) | ISOLATED SCAPHOCEPHALY |
| | (ORPHA:35093), POTOCKI-SHAFFER SYNDROME |
| | (ORPHA:52022), FRONTONASAL |
| | DYSPLASIA 2 (OMIM:613451), FRONTONASAL |
| | DYSPLASIA WITH ALOPECIA AND . . . |
| | (ORPHA:228390) |
| STAT1 | IMMUNODEFICIENCY 31A (OMIM:614892), |
| (6772) | IMMUNODEFICIENCY 31C |
| | (OMIM:614162), MYCOBACTERIAL AND |
| | VIRAL INFECTIONS, SUSC . . . |
| | (OMIM:613796), AUTOIMMUNE ENTEROPATHY |
| | AND ENDOCRINOPATH . . . |
| | (ORPHA:391487) |
| PHF21A | POTOCKI-SHAFFER SYNDROME (ORPHA:52022) |
| (51317) | |
| MKS1 | MECKEL SYNDROME (ORPHA:564), |
| (54903) | JOUBERT SYNDROME WITH OCULAR |
| | DEFECT (ORPHA:220493), JOUBERT |
| | SYNDROME (ORPHA:475), BARDET-BIEDL |
| | SYNDROME 13 (OMIM:615990), BARDET-BIEDL |
| | SYNDROME (ORPHA:110), |
| | MECKEL SYNDROME, TYPE 1 (OMIM:249000) |
| HIRA (7290) | 22Q11.2 DELETION SYNDROME (ORPHA:567) |
| NOD2 | SARCOIDOSIS, EARLY-ONSET (OMIM:609464), |
| (64127) | BLAU SYNDROME (ORPHA:90340), |
| | SYNOVITIS, GRANULOMATOUS, |
| | WITH UVEITIS A . . . (OMIM:186580) |
| SLC52A3 | RIBOFLAVIN TRANSPORTER DEFICIENCY |
| (113278) | (ORPHA:97229), BROWN-VIALETTO- |
| | VAN LAERE SYNDROME 1 (OMIM:211530), |
| | BULBAR PALSY, PROGRESSIVE, OF |
| | CHILDHOOD (OMIM:211500) |
| LRIG2 | OCHOA SYNDROME (ORPHA:2704), |
| (9860) | UROFACIAL SYNDROME 2 (OMIM:615112) |
| JAK2 (3717) | BUDD-CHIARI SYNDROME (ORPHA:131), |
| | ERYTHROCYTOSIS, FAMILIAL, 1 |
| | (OMIM:133100), POLYCYTHEMIA VERA |
| | (ORPHA:729), THROMBOCYTHEMIA 3 |
| | (OMIM:614521), POLYCYTHEMIA |
| | VERA (OMIM:263300), ESSENTIAL |
| | THROMBOCYTHEMIA (ORPHA:3318), |
| | FAMILIAL THROMBOCYTOSIS |
| | (ORPHA:71493), MYELOFIBROSIS (OMIM:254450) |
| ARL6 | RETINITIS PIGMENTOSA (ORPHA:791), |
| (84100) | RETINITIS PIGMENTOSA (OMIM:268000), |
| | RETINITIS PIGMENTOSA 55 (OMIM:613575), |
| | BARDET-BIEDL SYNDROME |
| | (ORPHA:110), BARDET-BIEDL |
| | SYNDROME 3 (OMIM:600151) |
| SLC2A10 | ARTERIAL TORTUOSITY SYNDROME |
| (81031) | (OMIM:208050) |
| ERCC8 | COCKAYNE SYNDROME A (OMIM:216400), |
| (1161) | UV-SENSITIVE SYNDROME 2 |
| | (OMIM:614621) |
| KLHL3 | PSEUDOHYPOALDOSTERONISM, |
| (26249) | TYPE IID (OMIM:614495) |
| TTC8 | RETINITIS PIGMENTOSA (ORPHA:791), |
| (123016) | BARDET-BIEDL SYNDROME 8 |
| | (OMIM:615985), RETINITIS PIGMENTOSA |
| | 51 (OMIM:613464), BARDET-BIEDL |
| | SYNDROME (ORPHA:110) |
| BMPR2 | PULMONARY HYPERTENSION, |
| (659) | PRIMARY, 1 (OMIM:178600), PULMONARY |
| | VENOOCCLUSIVE DISEASE (OMIM:265450) |
| FBN1 (2200) | GLAUCOMA-ECTOPIA-MICROSPHEROPHAKIA- |
| | STIFF . . . (ORPHA:2084), NEONATAL |
| | MARFAN SYNDROME (ORPHA:284979), |
| | ISOLATED ECTOPIA LENTIS |
| | (ORPHA:1885), MARFAN SYNDROME |
| | (OMIM:154700), FAMILIAL THORACIC |
| | AORTIC ANEURYSM AND AO . . . |
| | (ORPHA:91387), STIFF SKIN SYNDROME |
| | (OMIM:184900), ACROMICRIC DYSPLASIA |
| | (ORPHA:969), ECTOPIA LENTIS, |
| | ISOLATED (OMIM:129600), ACROMICRIC |
| | DYSPLASIA (OMIM:102370), WEILL- |
| | MARCHESANI SYNDROME, AUTOSOMAL |
| | DOM . . . (OMIM:608328), MASS |
| | SYNDROME (OMIM:604308), WEILL- |
| | MARCHESANI SYNDROME (ORPHA:3449), |
| | STIFF SKIN SYNDROME (ORPHA:2833), |
| | GELEOPHYSIC DYSPLASIA 2 |
| | (OMIM:614185) |
| NF1 (4763) | JUVENILE MYELOMONOCYTIC LEUKEMIA |
| | (OMIM:607785), NEUROFIBROMATOSIS, |
| | TYPE I (OMIM:162200), NEUROFIBROMATOSIS- |
| | NOONAN SYNDROME |
| | (ORPHA:638), NEUROFIBROMATOSIS, |
| | FAMILIAL SPINAL (OMIM:162210), WATSON |
| | SYNDROME (OMIM:193520), |
| | NEUROFIBROMATOSIS-NOONAN SYNDROME |
| | (OMIM:601321), 17Q11.2 MICRODUPLICATION |
| | SYNDROME (ORPHA:139474) |
| GLA (2717) | FABRY DISEASE (ORPHA:324), FABRY |
| | DISEASE (OMIM:301500) |
| ALMS1 | ALSTROM SYNDROME (OMIM:203800), |
| (7840) | ALSTRA-M SYNDROME (ORPHA:64) |
| ARHGAP31 | ADAMS-OLIVER SYNDROME (OMIM:100300), |
| (57514) | ADAMS-OLIVER SYNDROME |
| | (ORPHA:974) |
| SHPK | ISOLATED SEDOHEPTULOKINASE |
| (23729) | DEFICIENCY (ORPHA:440713) |
| KCNJ5 | FAMILIAL HYPERALDOSTERONISM |
| (3762) | TYPE III (ORPHA:251274), |
| | HYPERALDOSTERONISM, FAMILIAL, |
| | TYPE III (OMIM:613677), LONG QT |
| | SYNDROME 13 (OMIM:613485) |
| DGUOK | MITOCHONDRIAL DNA DEPLETION |
| (1716) | SYNDROME 3 ( . . . (OMIM:251880) |
| SCN2B | ATRIAL FIBRILLATION, FAMILIAL, |
| (6327) | 14 (OMIM:615378) |
| UFD1 | 22Q11.2 DELETION SYNDROME (ORPHA:567) |
| (7353) | |
| SCNN1B | PSEUDOHYPOALDOSTERONISM, TYPE I, |
| (6338) | AUTOSOM . . . (OMIM:264350), LIDDLE |
| | SYNDROME (ORPHA:526), LIDDLE |
| | SYNDROME (OMIM:177200), BRONCHIECTASIS |
| | WITH OR WITHOUT ELEVATED . . . |
| | (OMIM:211400) |
| PKHD1 | POLYCYSTIC KIDNEY DISEASE, AUTOSOMAL |
| (5314) | REC . . . (OMIM:263200), AUTOSOMAL |
| | RECESSIVE POLYCYSTIC KIDNEY |
| | DI . . . (ORPHA:731) |
| FGA (2243) | FAMILIAL HYPOFIBRINOGENEMIA |
| | (ORPHA:101041), FAMILIAL |
| | DYSFIBRINOGENEMIA (ORPHA:98881), |
| | AFIBRINOGENEMIA, |
| | CONGENITALHYPOFIBRINOGE . . . |
| | (OMIM:202400), AMYLOIDOSIS, FAMILIAL |
| | VISCERAL (OMIM:105200), FAMILIAL |
| | AFIBRINOGENEMIA (ORPHA:98880) |
| SCNN1G | PSEUDOHYPOALDOSTERONISM, TYPE I, |
| (6340) | AUTOSOM . . . (OMIM:264350), LIDDLE |
| | SYNDROME (ORPHA:526), BRONCHIECTASIS |
| | WITH OR WITHOUT ELEVATED . . . |
| | (OMIM:613071), LIDDLE SYNDROME |
| | (OMIM:177200) |
| TJP2 (9414) | CHOLESTASIS, PROGRESSIVE FAMILIAL |
| | INTRAH . . . (OMIM:615878), |
| | HYPERCHOLANEM IA, FAMILIAL |
| | (OMIM:607748) |
| CC2D2A | MECKEL SYNDROME (ORPHA:564), |
| (57545) | MECKEL SYNDROME, TYPE 6 (OMIM:612284), |
| | JOUBERT SYNDROME WITH HEPATIC |
| | DEFECT (ORPHA:1454), JOUBERT |
| | SYNDROME 9 (OMIM:612285), JOUBERT |
| | SYNDROME WITH OCULORENAL |
| | DEFECT (ORPHA:2318), COACH |
| | SYNDROME (OMIM:216360) |
| MAFB | MULTICENTRIC CARPO-TARSAL |
| (9935) | OSTEOLYSIS WIT . . . (ORPHA:2774), |
| | MULTICENTRIC CARPOTARSAL |
| | OSTEOLYSIS SYN D . . . (OMIM:166300), DUANE |
| | RETRACTION SYNDROME (ORPHA:233) |
| NR3C2 | HYPERTENSION, EARLY-ONSET, |
| (4306) | AUTOSOMAL DOM . . . (OMIM:605115), |
| | PSEUDOHYPOALDOSTERONISM, |
| | TYPE I, AUTOSOM . . . (OMIM:177735) |
| CCR6 | DIFFUSE CUTANEOUS SYSTEMIC |
| (1235) | SCLEROSIS (ORPHA:220393), LIMITED |
| | CUTANEOUS SYSTEMIC SCLEROSIS |
| | (ORPHA:220402) |
| FGFR2 | BENT BONE DYSPLASIA SYNDROME |
| (2263) | (OMIM:614592), ANTLEY-BIXLER |
| | SYNDROME (ORPHA:83), JACKSON- |
| | WEISS SYNDROME (OMIM:123150), CUTIS |
| | GYRATA-ACANTHOSIS NIGRICANS- |
| | CRANIO . . . (ORPHA:1555), PFEIFFER |
| | SYNDROME (OMIM:101600), APERT |
| | SYNDROME (ORPHA:87), CROUZON |
| | SYNDROME (OMIM:123500), SAETHRE- |
| | CHOTZEN SYNDROME (ORPHA:794), |
| | JACKSON-WEISS SYNDROME (ORPHA:1540), |
| | LACRIMOAURICULODENTODIGITAL |
| | SYNDROME (OMIM:149730), FGFR2- |
| | RELATED BENT BONE DYSPLASIA |
| | (ORPHA:313855), BEARE-STEVENSON |
| | CUTIS GYRATA SYNDROME |
| | (OMIM:123790), PFEIFFER SYNDROME |
| | TYPE 1 (ORPHA:93258), PFEIFFER |
| | SYNDROME TYPE 3 (ORPHA:93260), |
| | SAETHRE-CHOTZEN SYNDROME |
| | (OMIM:101400), ANTLEY-BIXLER |
| | SYNDROME WITHOUT GENITAL A . . . |
| | (OMIM:207410), PFEIFFER SYNDROME |
| | TYPE 2 (ORPHA:93259), FAMILIAL |
| | SCAPHOCEPHALY SYNDROME, |
| | MCGILLI . . . (OMIM:609579), GASTRIC |
| | CANCERGASTRIC CANCER, |
| | INTESTINAL . . . (OMIM:613659), FAMILIAL |
| | SCAPHOCEPHALY SYNDROME, |
| | MCGILLI . . . (ORPHA:168624), APERT SYNDROME |
| | (OMIM:101200), CROUZON DISEASE (ORPHA:207) |
| GNAS | PSEUDOPSEUDOHYPOPARATHYROIDISM |
| (2778) | (OMIM:612463), MCCUNE-ALBRIGHT |
| | SYNDROME (ORPHA:562), |
| | PSEUDOHYPOPARATHYROIDISM, TYPE IC |
| | (OMIM:612462), MCCUNE-ALBRIGHT |
| | SYNDROME (OMIM:174800), PITUITARY |
| | ADENOMA, GROWTH HORMONE-SECRET . . . |
| | (OMIM:102200), OSSEOUS |
| | HETEROPLASIA, PROGRESSIVE |
| | (OMIM:166350), CUSHING SYNDROME DUE TO |
| | MACRONODULAR ADR . . . |
| | (ORPHA:189427), ACTH-INDEPENDENT |
| | MACRONODULAR ADRENAL |
| | HY . . . (OMIM:219080), |
| | PSEUDOHYPOPARATHYROIDISM, |
| | TYPE IA (OMIM:103580), |
| | PSEUDOHYPOPARATHYROIDISM, TYPE |
| | IB (OMIM:603233), PROGRESSIVE |
| | OSSEOUS HETEROPLASIA (ORPHA:2762) |
| HSD11B2 | APPARENT MINERALOCORTICOID EXCESS |
| (3291) | (OMIM:218030) |
| SLC52A2 | BROWN-VIALETTO-VAN LAERE SYNDROME |
| (79581) | 2 (OMIM:614707), RIBOFLAVIN |
| | TRANSPORTER DEFICIENCY (ORPHA:97229) |
| NME1 | NEUROBLASTOMA (OMIM:256700) |
| (4830) | |
| PLIN1 | LIPODYSTROPHY, FAMILIAL PARTIAL, |
| (5346) | TYPE 4 (OMIM:613877), PLIN1-RELATED |
| | FAMILIAL PARTIAL LIPODYSTR . . . |
| | (ORPHA:280356) |
| DOCK6 | ADAMS-OLIVER SYNDROME (ORPHA:974), |
| (57572) | ADAMS-OLIVER SYNDROME 2 |
| | (OMIM:614219) |
| ADAMTSL4 | ISOLATED ECTOPIA LENTIS (ORPHA:1885), |
| (54507) | ECTOPIA LENTIS ET PUPILLAE |
| | (OMIM:225200), ECTOPIA LENTIS (OMIM:225100) |
| TNFSF15 | PRIMARY BILIARY CHOLANGITIS (ORPHA:186) |
| (9966) | |
| WNK4 | PSEUDOHYPOALDOSTERONISM, |
| (65266) | TYPE IIB (OMIM:614491) |
| NOTCH1 | ADAMS-OLIVER SYNDROME 5 (OMIM:616028), |
| (4851) | ADAMS-OLIVER SYNDROME |
| | (ORPHA:974), AORTIC VALVE |
| | DISEASE 1 (OMIM:109730) |
| TBX1 (6899) | TETRALOGY OF FALLOT (OMIM:187500), |
| | DIGEORGE SYNDROME (OMIM:188400), |
| | 22Q11.2 DELETION SYNDROME (ORPHA:567), |
| | VELOCARDIOFACIAL SYNDROME |
| | (OMIM:192430), CONOTRUNCAL HEART |
| | MALFORMATIONS (OMIM:217095), |
| | 22Q11.2 MICRODUPLICATION SYNDROME |
| | (ORPHA:1727) |
| SDHB | COWDEN-LIKE SYNDROME (OMIM:612359), |
| (6390) | GASTROINTESTINAL STROMAL |
| | TUMOR (OMIM:606764), |
| | PHEOCHROMOCYTOMA (OMIM:171300), |
| | PARAGANGLIOMAS 4 (OMIM:115310), |
| | CARNEY-STRATAKIS SYNDROME |
| | (ORPHA:97286), COWDEN SYNDROME |
| | (ORPHA:201), CARNEY-STRATAKIS |
| | SYNDROME (OMIM:606864), |
| | GASTROINTESTINAL STROMAL TUMOR |
| | (ORPHA:44890) |
| NOTCH3 | LATERAL MENINGOCELE SYNDROME |
| (4854) | (OMIM:130720), INFANTILE |
| | MYOFIBROMATOSIS (ORPHA:2591), |
| | CEREBRAL ARTERIOPATHY, AUTOSOMAL |
| | DOMINAN . . . (OMIM:125310), CADASIL |
| | (ORPHA:136), MYOFIBROMATOSIS, |
| | INFANTILE, 2 (OMIM:615293) |
| FOXF1 | CONGENITAL ALVEOLAR CAPILLARY |
| (2294) | DYSPLASIA (ORPHA:210122), ALVEOLAR |
| | CAPILLARY DYSPLASIA WITH MISALI . . . |
| | (OMIM:265380) |
| SDHC | GASTROINTESTINAL STROMAL TUMOR |
| (6391) | (OMIM:606764), PARAGANGLIOMAS 3 |
| | (OMIM:605373), CARNEY-STRATAKIS |
| | SYNDROME (ORPHA:97286), COWDEN |
| | SYNDROME (ORPHA:201), CARNEY- |
| | STRATAKIS SYNDROME (OMIM:606864), |
| | GASTROINTESTINAL STROMAL TUMOR |
| | (ORPHA:44890) |
| SDHD | PHEOCHROMOCYTOMA (OMIM:171300), |
| (6392) | COWDEN SYNDROME 3 (OMIM:615106), |
| | PARAGANGLIOMAS 1 (OMIM:168000), |
| | MITOCHONDRIAL COMPLEX II DEFICIENCY |
| | (OMIM:252011), CARNEY-STRATAKIS |
| | SYNDROME (ORPHA:97286), COWDEN |
| | SYNDROME (ORPHA:201), CARCINOID |
| | TUMORS, INTESTINAL (OMIM:114900), |
| | CARNEY-STRATAKIS SYNDROME |
| | (OMIM:606864) |
| PDE11A | PRIMARY PIGMENTED NODULAR |
| (50940) | ADRENOCORTICAL . . . (ORPHA:189439), |
| | PIGMENTED NODULAR ADRENOCORTICAL |
| | DISEASE . . . (OMIM:610475) |
| GP1BB | BERNARD-SOULIER SYNDROME |
| (2812) | (OMIM:231200), 22Q11.2 DELETION SYNDROME |
| | (ORPHA:567) |
| COL1A1 | EHLERS-DANLOS SYNDROME TYPE 2 |
| (1277) | (ORPHA:90318), OSTEOGENESIS |
| | IMPERFECTA, TYPE IV (OMIM:166220), |
| | EHLERS-DANLOS SYNDROME TYPE 1 |
| | (ORPHA:90309), CAFFEY DISEASE |
| | (OMIM:114000), OSTEOGENESIS IMPERFECTA, |
| | TYPE I (OMIM:166200), EHLERS-DANLOS |
| | SYNDROME, TYPE I (OMIM:130000), |
| | OSTEOGENESIS IMPERFECTA, TYPE IIA |
| | (OMIM:166210), CAFFEY DISEASE |
| | (ORPHA:1310), EHLERS-DANLOS SYNDROME |
| | TYPE 7A (ORPHA:99875), EHLERS- |
| | DANLOS SYNDROME, TYPE VII, AUTOSO . . . |
| | (OMIM:130060), OSTEOGENESIS |
| | IMPERFECTA, TYPE III (OMIM:259420), |
| | DERMATOFIBROSARCOMA |
| | PROTUBERANS (ORPHA:31112) |
| FOXE3 | ANTERIOR SEGMENT MESENCHYMAL |
| (2301) | DYSGENESIS (OMIM:107250), APHAKIA, |
| | CONGENITAL PRIMARY (OMIM:610256), |
| | CONGENITAL PRIMARY APHAKIA |
| | (ORPHA:83461), FAMILIAL THORACIC |
| | AORTIC ANEURYSM AND AO . . . |
| | (ORPHA:91387) |
| MPL (4352) | POLYCYTHEMIA VERA (ORPHA:729), |
| | AMEGAKARYOCYTIC |
| | THROMBOCYTOPENIA, CONGEN . . . |
| | (OMIM:604498), THROMBOCYTHEMIA 2 |
| | (OMIM:601977), ESSENTIAL |
| | THROMBOCYTHEMIA (ORPHA:3318), |
| | CONGENITAL |
| | AMEGAKARYOCYTIC THROMBOCYTOPE . . . |
| | (ORPHA:3319), FAMILIAL |
| | THROMBOCYTOSIS (ORPHA:71493), |
| | MYELOFIBROSIS (OMIM:254450) |
| COL3A1 | EHLERS-DANLOS SYNDROME, TYPE III |
| (1281) | (OMIM:130020), EHLERS-DANLOS |
| | SYNDROME, VASCULAR TYPE (ORPHA:286), |
| | EHLERS-DANLOS SYNDROME, TYPE |
| | IV, AUTOSOM . . . (OMIM:130050), |
| | ACROGERIA (ORPHA:2500) |
| NPHP1 | JOUBERT SYNDROME WITH RENAL DEFECT |
| (4867) | (ORPHA:220497), SENIOR-LOKEN |
| | SYNDROME 1 (OMIM:266900), JOUBERT |
| | SYNDROME 4 (OMIM:609583), BARDET- |
| | BIEDL SYNDROME (ORPHA:110), |
| | NEPHRONOPHTHISIS 1 (OMIM:256100), SENIOR- |
| | LOKEN SYNDROME (ORPHA:3156) |
| VHL (7428) | PHEOCHROMOCYTOMA (OMIM:171300), |
| | RENAL CELL CARCINOMA, |
| | NONPAPILLARY (OMIM:144700), VON |
| | HIPPEL-LINDAU SYNDROME (OMIM:193300), |
| | VON HIPPEL-LINDAU DISEASE (ORPHA:892), |
| | ERYTHROCYTOSIS, FAMILIAL, 2 |
| | (OMIM:263400) |
| CUL3 | PSEUDOHYPOALDOSTERONISM, |
| (8452) | TYPE IIE (OMIM:614496) |
| COL4A3 | HEMATURIA, BENIGN FAMILIAL |
| (1285) | (OMIM:141200), ALPORT SYNDROME, |
| | AUTOSOMAL RECESSIVE (OMIM:203780), |
| | ALPORT SYNDROME, AUTOSOMAL |
| | DOMINANT (OMIM:104200) |
| COL4A4 | ALPORT SYNDROME, AUTOSOMAL |
| (1286) | RECESSIVE (OMIM:203780) |
| COL4A5 | ALPORT SYNDROME, X-LINKED |
| (1287) | (OMIM:301050) |
| CACNA1D | SINOATRIAL NODE DYSFUNCTION |
| (776) | AND DEAFNESS (OMIM:614896), |
| | ALDOSTERONE-PRODUCING ADENOMA |
| | WITH SEIZU . . . (ORPHA:369929), |
| | PRIMARY ALDOSTERONISM, SEIZURES, |
| | AND NEU . . . (OMIM:615474) |
| COL5A1 | EHLERS-DANLOS SYNDROME TYPE 2 |
| (1289) | (ORPHA:90318), EHLERS-DANLOS |
| | SYNDROME TYPE 1 (ORPHA:90309), |
| | EHLERS-DANLOS SYNDROME, VASCULAR |
| | TYPE (ORPHA:286), EHLERS-DANLOS |
| | SYNDROME, TYPE I (OMIM:130000) |
| COL5A2 | EHLERS-DANLOS SYNDROME TYPE 2 |
| (1290) | (ORPHA:90318), EHLERS-DANLOS |
| | SYNDROME TYPE 1 (ORPHA:90309), |
| | EHLERS-DANLOS SYNDROME, TYPE I |
| | (OMIM:130000) |
| IFT27 | BARDET-BIEDL SYNDROME (ORPHA:110), |
| (11020) | BARDET-BIEDL SYNDROME 19 |
| | (OMIM:615996) |
| FMO3 | TRIMETHYLAMINURIA (OMIM:602079) |
| (2328) | |
| RPGRIP1L | MECKEL SYNDROME (ORPHA:564), |
| (23322) | JOUBERT SYNDROME 7 (OMIM:611560), |
| | JOUBERT SYNDROME WITH HEPATIC |
| | DEFECT (ORPHA:1454), JOUBERT |
| | SYNDROME WITH RENAL DEFECT |
| | (ORPHA:220497), MECKEL SYNDROME, TYPE |
| | 5 (OMIM:611561), COACH SYNDROME |
| | (OMIM:216360) |
| FMR1 | XQ27.3Q28 DUPLICATION SYNDROME |
| (2332) | (ORPHA:261483), FRAGILE X |
| | TREMOR/ATAXIA SYNDROME |
| | (OMIM:300623), FRAGILE X-ASSOCIATED |
| | TREMOR/ATAXIA SYNDR . . . (ORPHA:93256), |
| | FRAGILE X SYNDROME (ORPHA:908), |
| | FRAGILE X MENTAL RETARDATION |
| | SYNDROME (OMIM:300624), PREMATURE |
| | OVARIAN FAILURE 1 (OMIM:311360) |
| FN1 (2335) | FIBRONECTIN GLOMERULOPATHY |
| | (ORPHA:84090), GLOMERULOPATHY WITH |
| | FIBRONECTIN DEPOSITS . . . (OMIM:601894) |
| COMT | 22Q11.2 DELETION SYNDROME (ORPHA:567) |
| (1312) | |
| OFD1 | RETINITIS PIGMENTOSA (ORPHA:791), |
| (8481) | RETINITIS PIGMENTOSA 23 |
| | (OMIM:300424), PRIMARY CILIARY DYSKINESIA |
| | (ORPHA:244), OROFACIODIGITAL |
| | SYNDROME TYPE 1 (ORPHA:2750), |
| | OROFACIODIGITAL SYNDROME I |
| | (OMIM:311200), SIMPSON-GOLABI-BEHMEL |
| | SYNDROME, TYPE 2 (OMIM:300209), |
| | JOUBERT SYNDROME 10 (OMIM:300804) |
| MLX (6945) | TAKAYASU ARTERITIS (ORPHA:3287) |
| SH2B3 | ERYTHROCYTOSIS, FAMILIAL, 1 |
| (10019) | (OMIM:133100), THROMBOCYTHEMIA, |
| | ESSENTIAL (OMIM:187950), ESSENTIAL |
| | THROMBOCYTHEMIA (ORPHA:3318), |
| | MYELOFIBROSIS (OMIM:254450) |
| CLIP2 | WILLIAMS SYNDROME (ORPHA:904) |
| (7461) | |
| DLL4 | ADAMS-OLIVER SYNDROME |
| (54567) | (ORPHA:974), APLASIA CUTIS CONGENITA |
| | (ORPHA:1114), ADAMS-OLIVER |
| | SYNDROME 6 (OMIM:616589) |
| CALR (811) | THROMBOCYTHEMIA, ESSENTIAL |
| | (OMIM:187950), ESSENTIAL |
| | THROMBOCYTHEMIA (ORPHA:3318), |
| | MYELOFIBROSIS (OMIM:254450) |
| INPP5E | JOUBERT SYNDROME WITH OCULAR |
| (56623) | DEFECT (ORPHA:220493), JOUBERT |
| | SYNDROME 1 (OMIM:213300), JOUBERT |
| | SYNDROME WITH HEPATIC DEFECT |
| | (ORPHA:1454), JOUBERT SYNDROME |
| | (ORPHA:475), MENTAL RETARDATION, |
| | TRUNCAL OBESITY, RET . . . (OMIM:610156) |
| SMARCAL1 | IMMUNOOSSEOUS DYSPLASIA, |
| (50485) | SCHIMKE TYPE (OMIM:242900), SCHIMKE |
| | IMMUNO-OSSEOUS DYSPLASIA (ORPHA:1830) |
| CEP290 | MECKEL SYNDROME (ORPHA:564), |
| (80184) | LEBER CONGENITAL AMAUROSIS |
| | (ORPHA:65), JOUBERT SYNDROME 5 |
| | (OMIM:610188), SENIOR-LOKEN SYNDROME |
| | 6 (OMIM:610189), MECKEL SYNDROME, TYPE 4 |
| | (OMIM:611134), BARDET-BIEDL |
| | SYNDROME 14 (OMIM:615991), JOUBERT |
| | SYNDROME WITH OCULORENAL |
| | DEFECT (ORPHA:2318), LEBER CONGENITAL |
| | AMAUROSIS 10 (OMIM:611755), |
| | BARDET-BIEDL SYNDROME (ORPHA:110), |
| | SENIOR-LOKEN SYNDROME |
| | (ORPHA:3156) |
| LZTFL1 | BARDET-BIEDL SYNDROME 17 |
| (54585) | (OMIM:615994), BARDET-BIEDL SYNDROME |
| | (ORPHA:110) |
| WRN (7486) | WERNER SYNDROME (OMIM:277700), |
| | WERNER SYNDROME (ORPHA:902) |
| WT1 (7490) | ANIRIDIA (OMIM:106210), WILMS |
| | TUMOR, ANIRIDIA, GENITOURINARY ANO . . . |
| | (OMIM:194072), DESMOPLASTIC SMALL |
| | ROUND CELL TUMOR (ORPHA:83469), |
| | FRASIER SYNDROME (OMIM:136680), |
| | DENYS-DRASH SYNDROME (OMIM:194080), |
| | MESOTHELIOMA, MALIGNANT (OMIM:156240), |
| | NEPHROBLASTOMA (ORPHA:654), |
| | WAGR SYNDROME (ORPHA:893), WILMS |
| | TUMOR 1 (OMIM:194070), NEPHROTIC |
| | SYNDROME, EARLY-ONSET, |
| | WITH DI . . . (OMIM:256370) |
| BBIP1 | BARDET-BIEDL SYNDROME 18 |
| (92482) | (OMIM:615995), BARDET-BIEDL SYNDROME |
| | (ORPHA:110) |
| ITGA8 | RENAL HYPODYSPLASIA/APLASIA 1 |
| (8516) | (OMIM:191830), RENAL AGENESIS, |
| | BILATERAL (ORPHA:1848) |
| IKBKAP | NEUROPATHY, HEREDITARY SENSORY |
| (8518) | AND AUTON . . . (OMIM:223900), FAMILIAL |
| | DYSAUTONOMIA (ORPHA:1764) |
| FUZ (80199) | NEURAL TUBE DEFECTS |
| | (OMIM:182940), CAUDAL REGRESSION SEQUENCE |
| | (ORPHA:3027) |
| BAZ1B | WILLIAMS SYNDROME (ORPHA:904) |
| (9031) | |
| POR (5447) | CONGENITAL ADRENAL HYPERPLASIA |
| | DUE TO CY . . . (ORPHA:95699), |
| | DISORDERED STEROIDOGENESIS DUE TO |
| | CYTOCH . . . (OMIM:613571), ANTLEY- |
| | BIXLER SYNDROME WITH GENITAL ANOM . . . |
| | (OMIM:201750), ANTLEY-BIXLER |
| | SYNDROME WITHOUT GENITAL |
| | A . . . (OMIM:207410) |
| ABCB6 | DYSCHROMATOSIS UNIVERSALIS |
| (10058) | HEREDITARIA 3 (OMIM:615402), |
| | DYSCHROMATOSIS UNIVERSALIS |
| | (ORPHA:241), FAMILIAL |
| | PSEUDOHYPERKALEMIA (ORPHA:90044), |
| | MICROPHTHALMIA, ISOLATED, WITH |
| | COLOBOMA . . . (OMIM:614497) |
| POU2AF1 | PRIMARY BILIARY CHOLANGITIS (ORPHA:186) |
| (5450) | |
| APOA1 | AMYLOIDOSIS, FAMILIAL |
| (335) | VISCERAL (OMIM:105200), |
| | HYPOALPHALIPOPROTEINEMIA, |
| | PRIMARY (OMIM:604091), APOLIPOPROTEIN A-I |
| | DEFICIENCY (ORPHA:425) |
| AIP (9049) | PITUITARY ADENOMA, PROLACTIN- |
| | SECRETING (OMIM:600634), PITUITARY |
| | ADENOMA, GROWTH HORMONE-SECRET . . . |
| | (OMIM:102200), PITUITARY |
| | ADENOMA, ACTH-SECRETING (OMIM:219090), |
| | PROLACTINOMA (ORPHA:2965), |
| | ACROMEGALY (ORPHA:963) |
| CAV1 (857) | PARTIAL LIPODYSTROPHY, CONGENITAL |
| | CATARA . . . (OMIM:606721), DIFFUSE |
| | CUTANEOUS SYSTEMIC SCLEROSIS |
| | (ORPHA:220393), LIMITED CUTANEOUS |
| | SYSTEMIC SCLEROSIS (ORPHA:220402), |
| | PULMONARY HYPERTENSION, |
| | PRIMARY, 3 (OMIM:615343), BERARDINELLI- |
| | SEIP CONGENITAL LIPODYSTROP . . . |
| | (ORPHA:528), LIPODYSTROPHY, CONGENITAL |
| | GENERALIZED, T . . . (OMIM:612526) |
| BBS5 | BARDET-BIEDL SYNDROME 5 (OMIM:615983), |
| (129880) | BARDET-BIEDL SYNDROME |
| | (ORPHA:110) |
| REST | NEPHROBLASTOMA (ORPHA:654) |
| (5978) | |
| RET (5979) | RENAL HYPODYSPLASIA/APLASIA 1 |
| | (OMIM:191830), HIRSCHSPRUNG DISEASE |
| | (ORPHA:388), MULTIPLE ENDOCRINE |
| | NEOPLASIA, TYPE IIA (OMIM:171400), |
| | HADDAD SYNDROME (ORPHA:99803), |
| | MULTIPLE ENDOCRINE NEOPLASIA, TYPE |
| | IIB (OMIM:162300), PHEOCHROMOCYTOMA |
| | (OMIM:171300), CENTRAL |
| | HYPOVENTILATION SYNDROME, |
| | CONGEN . . . (OMIM:209880), THYROID |
| | CARCINOMA, FAMILIAL MEDULLARY |
| | (OMIM:155240), RENAL AGENESIS, |
| | BILATERAL (ORPHA:1848) |
| CPOX | COPROPORPHYRIA, HEREDITARY |
| (1371) | (OMIM:121300), HEREDITARY |
| | COPROPORPHYRIA (ORPHA:79273) |
| NR3C1 | GLUCOCORTICOID RESISTANCE |
| (2908) | (ORPHA:786), GLUCOCORTICOID RESISTANCE, |
| | GENERALIZED (OMIM:615962) |
| PPARG | CAROTID INTIMAL MEDIAL THICKNESS |
| (5468) | 1 (OMIM:609338), LIPODYSTROPHY, |
| | FAMILIAL PARTIAL, TYPE 3 (OMIM:604367), |
| | OBESITY (OMIM:601665), |
| | BERARDINELLI-SEIP CONGENITAL |
| | LIPODYSTROP . . . (ORPHA:528) |
| RFC2 | WILLIAMS SYNDROME (ORPHA:904) |
| (5982) | |
| GTF2IRD1 | WILLIAMS SYNDROME (ORPHA:904) |
| (9569) | |
| IDUA (3425) | HURLER-SCHEIE SYNDROME |
| | (ORPHA:93476), SCHEIE SYNDROME |
| | (OMIM:607016), HURLER SYNDROME |
| | (ORPHA:93473), SCHEIE SYNDROME |
| | (ORPHA:93474), HURLER SYNDROME |
| | (OMIM:607014), HURLER-SCHEIE |
| | SYNDROME (OMIM:607015) |
| SERPINA6 | CORTICOSTEROID-BINDING |
| (866) | GLOBULIN DEFICIEN . . . (OMIM:611489) |
| EDA (1896) | X-LINKED HYPOHIDROTIC ECTODERMAL |
| | DYSPLAS . . . (ORPHA:181), TOOTH |
| | AGENESIS, SELECTIVE, X-LINKED, 1 |
| | (OMIM:313500), OLIGODONTIA |
| | (ORPHA:99798), ECTODERMAL |
| | DYSPLASIA 1, HYPOHIDROTIC, X- . . . |
| | (OMIM:305100) |
| CBS (875) | HOMOCYSTINURIA DUE TO |
| | CYSTATHIONINE BETA . . . |
| | (OMIM:236200), CLASSIC |
| | HOMOCYSTINURIA (ORPHA:394) |
| JMJD1C | 22Q11.2 DELETION SYNDROME (ORPHA:567) |
| (221037) | |
| ABCC6 | PSEUDOXANTHOMA ELASTICUM, FORME |
| (368) | FRUSTEPS . . . (OMIM:177850), ARTERIAL |
| | CALCIFICATION, GENERALIZED, OF . . . |
| | (OMIM:614473), PSEUDOXANTHOMA |
| | ELASTICUM (ORPHA:758), PSEUDOXANTHOMA |
| | ELASTICUM (OMIM:264800) |
| WDPCP | MECKEL SYNDROME (ORPHA:564), HEART |
| (51057) | DEFECT-TONGUE HAMARTOMA- |
| | POLYSYNDAC . . . (ORPHA:1338), BARDET- |
| | BIEDL SYNDROME (ORPHA:110), |
| | CONGENITAL HEART DEFECTS, HAMARTOMAS |
| | OF . . . (OMIM:217085) |
| CEP164 | NEPHRONOPHTHISIS 15 (OMIM:614845), |
| (22897) | SENIOR-LOKEN SYNDROME |
| | (ORPHA:3156) |
| CLDN1 | ICHTHYOSIS-HYPOTRICHOSIS- |
| (9076) | SCLEROSING CHOL . . . (ORPHA:59303), |
| | ICHTHYOSIS, LEUKOCYTE VACUOLES, |
| | ALOPECIA . . . (OMIM:607626) |
| TNFRSF11B | PAGET DISEASE OF BONE 5, JUVENILE-ONSET |
| (4982) | (OMIM:239000), JUVENILE PAGET |
| | DISEASE (ORPHA:2801) |
| BBS10 | BARDET-BIEDL SYNDROME 10 |
| (79738) | (OMIM:615987), BARDET-BIEDL SYNDROME |
| | (ORPHA:110) |
| WDR19 | SHORT-RIB THORACIC DYSPLASIA |
| (57728) | 5 WITH OR W . . . (OMIM:614376), |
| | CRANIOECTODERMAL DYSPLASIA 4 |
| | (OMIM:614378), JEUNE SYNDROME |
| | (ORPHA:474), SENIOR-LOKEN |
| | SYNDROME 8 (OMIM:616307), |
| | CRANIOECTODERMAL DYSPLASIA |
| | (ORPHA:1515), SENIOR-LOKEN SYNDROME |
| | (ORPHA:3156) |
| PPP1R3A | LIPODYSTROPHY, FAMILIAL PARTIAL, |
| (5506) | TYPE 3 (OMIM:604367), DIABETES |
| | MELLITUS, NONINSULIN-DEPENDENT |
| | (OMIM:125853) |
| TGFB2 | LOEYS-DIETZ SYNDROME, TYPE 4 |
| (7042) | (OMIM:614816), FAMILIAL THORACIC AORTIC |
| | ANEURYSM AND AO . . . (ORPHA:91387) |
| TGFB3 | ARRHYTHMOGENIC RIGHT VENTRICULAR |
| (7043) | DYSPLAS . . . (OMIM:107970), LOEYS- |
| | DIETZ SYNDROME 5 (OMIM:615582), FAMILIAL |
| | THORACIC AORTIC ANEURYSM |
| | AND AO . . . (ORPHA:91387) |
| TGFBR1 | LOEYS-DIETZ SYNDROME (ORPHA:60030), |
| (7046) | FAMILIAL THORACIC AORTIC |
| | ANEURYSM AND AO . . . (ORPHA:91387) |
| TGFBR2 | LOEYS-DIETZ SYNDROME 2 |
| (7048) | (OMIM:610168), ESOPHAGEAL |
| | CANCERESOPHAGEAL SQUAMOUS CEL . . . |
| | (OMIM:133239), LOEYS-DIETZ |
| | SYNDROME (ORPHA:60030), SQUAMOUS |
| | CELL CARCINOMA OF ESOPHAGUS |
| | (ORPHA:99977), FAMILIAL THORACIC |
| | AORTIC ANEURYSM AND AO . . . |
| | (ORPHA:91387), COLORECTAL CANCER, |
| | HEREDITARY NONPOLYPOS . . . |
| | (OMIM:614331) |
| MFAP5 | AORTIC ANEURYSM, FAMILIAL THORACIC 9 |
| (8076) | (OMIM:616166), FAMILIAL THORACIC |
| | AORTIC ANEURYSM AND AO . . . (ORPHA:91387) |
| MLXIPL | WILLIAMS-BEUREN SYNDROME (OMIM:194050) |
| (51085) | |
| USP8 | CUSHING DISEASE (ORPHA:96253) |
| (9101) | |
| LIMK1 | WILLIAMS SYNDROME (ORPHA:904) |
| (3984) | |
| NPHP3 | NEPHRONOPHTHISIS 3 (OMIM:604387), |
| (27031) | RENAL-HEPATIC-PANCREATIC |
| | DYSPLASIA (OMIM:208540), MECKEL |
| | SYNDROME, TYPE 7 (OMIM:267010), |
| | SENIOR-LOKEN SYNDROME (ORPHA:3156) |
| GTF2I | WILLIAMS SYNDROME (ORPHA:904) |
| (2969) | |
| THPO | THROMBOCYTHEMIA, ESSENTIAL (OMIM:187950), |
| (7066) | FAMILIAL THROMBOCYTOSIS |
| | (ORPHA:71493) |
| MMEL1 | PRIMARY BILIARY CHOLANGITIS (ORPHA:186) |
| (79258) | |
| TRNC | MITOCHONDRIAL MYOPATHY, |
| (4511) | ENCEPHALOPATHY, . . . (OMIM:540000) |
| COX1 | MELAS (ORPHA:550), LEBER HEREDITARY |
| (4512) | OPTIC NEUROPATHY (ORPHA:104), |
| | MITOCHONDRIAL MYOPATHY, |
| | ENCEPHALOPATHY, . . . (OMIM:540000) |
| LMNA | HUTCHINSON-GILFORD PROGERIA |
| (4000) | SYNDROME (ORPHA:740), MUSCULAR |
| | DYSTROPHY, CONGENITAL, LMNA-REL . . . |
| | (OMIM:613205), MANDIBULOACRAL |
| | DYSPLASIA WITH TYPE A LIP . . . |
| | (OMIM:248370), FAMILIAL PARTIAL |
| | LIPODYSTROPHY, KA-BBERLI . . . |
| | (ORPHA:79084), MUSCULAR DYSTROPHY, LIMB- |
| | GIRDLE, TYPE 1B (OMIM:159001), EMERY- |
| | DREIFUSS MUSCULAR DYSTROPHY 3, |
| | AUT . . . (OMIM:616516), DILATED |
| | CARDIOMYOPATHY-HYPERGONADOTROPIC . . . |
| | (ORPHA:2229), EMERY-DREIFUSS |
| | MUSCULAR DYSTROPHY 2, AUT . . . |
| | (OMIM:181350), ATYPICAL WERNER |
| | SYNDROME (ORPHA:79474), HEART-HAND |
| | SYNDROME, SLOVENIAN TYPE |
| | (OMIM:610140), CHARCOT-MARIE-TOOTH |
| | DISEASE, AXONAL, TYP . . . (OMIM:605588), |
| | FAMILIAL PARTIAL LIPODYSTROPHY, |
| | DUNNIGAN . . . (ORPHA:2348), |
| | CARDIOMYOPATHY, |
| | DILATED, 1A (OMIM:115200), |
| | MANDIBULOACRAL DYSPLASIA WITH |
| | TYPE A LIP . . . (ORPHA:90153), LMNA- |
| | RELATED CARDIOCUTANEOUS PROGERIA |
| | SY . . . (ORPHA:363618), |
| | LIPODYSTROPHY, FAMILIAL PARTIAL, |
| | TYPE 2 (OMIM:151660), CONGENITAL |
| | MUSCULAR DYSTROPHY DUE TO LMN . . . |
| | (ORPHA:157973), CARDIOMYOPATHY, |
| | DILATED, WITH HYPERGONAD . . . |
| | (OMIM:212112), HUTCHINSON-GILFORD |
| | PROGERIA SYNDROME (OMIM:176670), |
| | LAMINOPATHY TYPE DECAUDAIN- |
| | VIGOUROUX (ORPHA:137871), |
| | RESTRICTIVE DERMOPATHY, LETHAL |
| | (OMIM:275210) |
| SEC24C | 22Q11.2 DELETION SYNDROME (ORPHA:567) |
| (9632) | |
| COX2 | MELAS (ORPHA:550), MITOCHONDRIAL |
| (4513) | MYOPATHY, ENCEPHALOPATHY, . . . |
| | (OMIM:540000) |
| COX3 | MELAS (ORPHA:550), LEBER |
| (4514) | HEREDITARY OPTIC NEUROPATHY (ORPHA:104), |
| | LEBER OPTIC ATROPHY (OMIM:535000), |
| | MITOCHONDRIAL MYOPATHY, |
| | ENCEPHALOPATHY, . . . (OMIM:540000) |
| EGFR | LUNG CANCERALVEOLAR CELL |
| (1956) | CARCINOMA, INCL . . . (OMIM:211980), |
| | INFLAMMATORY SKIN AND BOWEL |
| | DISEASE, NEO . . . (OMIM:616069) |
| ARVCF | 22Q11.2 DELETION SYNDROME (ORPHA:567) |
| (421) | |
| SUGCT | GLUTARIC ACIDURIA III (OMIM:231690) |
| (79783) | |
| GUCY1A3 | MOYAMOYA DISEASE 6 WITH |
| (2982) | ACHALASIA (OMIM:615750) |
| CYTB | LEBER HEREDITARY OPTIC NEUROPATHY |
| (4519) | (ORPHA:104), LEBER OPTIC ATROPHY |
| | (OMIM:535000), MITOCHONDRIAL |
| | MYOPATHY, ENCEPHALOPATHY, . . . |
| | (OMIM:540000) |
| LMX1B | NAIL-PATELLA SYNDROME (OMIM:161200), |
| (4010) | NAIL-PATELLA SYNDROME |
| | (ORPHA:2614) |
| TRIM32 | BARDET-BIEDL SYNDROME 11 |
| (22954) | (OMIM:615988), AUTOSOMAL RECESSIVE LIMB- |
| | GIRDLE MUSCULAR . . . (ORPHA:1878), |
| | BARDET-BIEDL SYNDROME (ORPHA:110), |
| | MUSCULAR DYSTROPHY, LIMB-GIRDLE, |
| | TYPE 2H (OMIM:254110) |
| BBS7 | BARDET-BIEDL SYNDROME 7 |
| (55212) | (OMIM:615984), BARDET-BIEDL SYNDROME |
| | (ORPHA:110) |
| VANGL1 | SACRAL DEFECT WITH ANTERIOR |
| (81839) | MENINGOCELE (OMIM:600145), CAUDAL |
| | REGRESSION SEQUENCE (ORPHA:3027) |
| PDE8B | PRIMARY PIGMENTED NODULAR |
| (8622) | ADRENOCORTICAL . . . (ORPHA:189439), |
| | STRIATAL DEGENERATION, AUTOSOMAL |
| | DOMINAN . . . (OMIM:609161), |
| | PIGMENTED NODULAR ADRENOCORTICAL |
| | DISEASE . . . (OMIM:614190), |
| | AUTOSOMAL DOMINANT STRIATAL |
| | NEURODEGENER . . . (ORPHA:228169) |
| LOX (4015) | FAMILIAL THORACIC AORTIC |
| | ANEURYSM AND AO . . . (ORPHA:91387) |
| UTP4 | NORTH AMERICAN INDIAN CHILDHOOD |
| (84916) | CIRRHOSI . . . (OMIM:604901) |
| ND1 (4535) | ISOLATED COMPLEX I DEFICIENCY |
| | (ORPHA:2609), MELAS (ORPHA:550), LEBER |
| | HEREDITARY OPTIC NEUROPATHY |
| | (ORPHA:104), LEBER OPTIC ATROPHY |
| | (OMIM:535000), MITOCHONDRIAL |
| | MYOPATHY, ENCEPHALOPATHY, . . . |
| | (OMIM:540000) |
| ARMC5 | ACTH-INDEPENDENT MACRONODULAR |
| (79798) | ADRENAL HY . . . (OMIM:615954), CUSHING |
| | SYNDROME DUE TO MACRONODULAR |
| | ADR . . . (ORPHA:189427) |
| XPNPEP3 | NEPHRONOPHTHISIS-LIKE NEPHROPATHY |
| (63929) | 1 (OMIM:613159) |
| IQCB1 | LEBER CONGENITAL AMAUROSIS (ORPHA:65), |
| (9657) | SENIOR-LOKEN SYNDROME 5 |
| | (OMIM:609254), SENIOR-LOKEN |
| | SYNDROME (ORPHA:3156) |
| ND4 (4538) | MELAS (ORPHA:550), LEBER HEREDITARY |
| | OPTIC NEUROPATHY (ORPHA:104), |
| | LEBER OPTIC ATROPHY (OMIM:535000) |
| ND5 (4540) | MERRF (ORPHA:551), MELAS |
| | (ORPHA:550), LEBER HEREDITARY OPTIC |
| | NEUROPATHY (ORPHA:104), LEBER |
| | OPTIC ATROPHY (OMIM:535000), |
| | MITOCHONDRIAL MYOPATHY, |
| | ENCEPHALOPATHY, . . . (OMIM:540000) |
| RBPJ (3516) | ADAMS-OLIVER SYNDROME 3 (OMIM:614814), |
| | ADAMS-OLIVER SYNDROME |
| | (ORPHA:974) |
| ND6 (4541) | MELAS (ORPHA:550), LEBER HEREDITARY |
| | OPTIC NEUROPATHY (ORPHA:104), |
| | LEBER OPTIC ATROPHY (OMIM:535000), |
| | MITOCHONDRIAL MYOPATHY, |
| | ENCEPHALOPATHY, . . . (OMIM:540000) |
| DYRK1B | ABDOMINAL OBESITY-METABOLIC |
| (9149) | SYNDROME 3 (OMIM:615812) |
| PRKACA | PRIMARY PIGMENTED NODULAR |
| (5566) | ADRENOCORTICAL . . . (ORPHA:189439), |
| | PIGMENTED NODULAR ADRENOCORTICAL |
| | DISEASE . . . (OMIM:615830) |
| PRKAR1A | PIGMENTED NODULAR ADRENOCORTICAL |
| (5573) | DISEASE . . . (OMIM:610489), THYROID |
| | CANCER, NONMEDULLARY, 1 (OMIM:188550), |
| | MYXOMA, INTRACARDIAC |
| | (OMIM:255960), PRIMARY PIGMENTED |
| | NODULAR ADRENOCORTICAL . . . |
| | (ORPHA:189439), CARNEY COMPLEX, TYPE 1 |
| | (OMIM:160980), ACRODYSOSTOSIS |
| | WITH MULTIPLE HORMONE RES . . . |
| | (ORPHA:280651), ACRODYSOSTOSIS |
| | (ORPHA:950), ACRODYSOSTOSIS 1, |
| | WITH OR WITHOUT HORMON . . . |
| | (OMIM:101800), CARNEY COMPLEX (ORPHA:1359) |
| TRNE | MATERNALLY-INHERITED DIABETES |
| (4556) | AND DEAFNE . . . (ORPHA:225) |
| TRNF | MERRF (ORPHA:551), MELAS (ORPHA:550), |
| (4558) | MYOCLONIC EPILEPSY ASSOCIATED |
| | WITH RAGGE . . . (OMIM:545000), |
| | MITOCHONDRIAL MYOPATHY, |
| | ENCEPHALOPATHY, . . . (OMIM:540000) |
| CTGF | DIFFUSE CUTANEOUS SYSTEMIC |
| (1490) | SCLEROSIS (ORPHA:220393), LIMITED |
| | CUTANEOUS SYSTEMIC SCLEROSIS |
| | (ORPHA:220402) |
| TRNH | MERRF (ORPHA:551), MELAS (ORPHA:550) |
| (4564) | |
| PAX2 | RENAL HYPODYSPLASIA/APLASIA 1 |
| (5076) | (OMIM:191830), PAPILLORENAL SYNDROME |
| | (OMIM:120330), RENAL COLOBOMA |
| | SYNDROME (ORPHA:1475), FOCAL |
| | SEGMENTAL GLOMERULOSCLEROSIS |
| | 7 (OMIM:616002) |
| CTLA4 | AUTOIMMUNE LYMPHOPROLIFERATIVE |
| (1493) | SYNDROME, . . . (OMIM:616100), SÃ‰ZARY |
| | SYNDROME (ORPHA:3162), GRANULOMATOSIS |
| | WITH POLYANGIITIS |
| | (ORPHA:900), CLASSIC MYCOSIS |
| | FUNGOIDES (ORPHA:2584) |
| ELN (2006) | WILLIAMS SYNDROME (ORPHA:904), |
| | SUPRAVALVULAR AORTIC STENOSIS |
| | (OMIM:185500), CUTIS LAXA, AUTOSOMAL |
| | DOMINANT 1 (OMIM:123700), |
| | SUPRAVALVULAR AORTIC STENOSIS |
| | (ORPHA:3193), AUTOSOMAL DOMINANT |
| | CUTIS LAXA (ORPHA:90348), WILLIAMS- |
| | BEUREN SYNDROME (OMIM:194050) |
| TRNK | MATERNALLY-INHERITED DIABETES AND |
| (4566) | DEAFNE . . . (ORPHA:225), MERRF |
| | (ORPHA:551), MATERNALLY-INHERITED |
| | CARDIOMYOPATHY AND . . . |
| | (ORPHA:1349), MYOCLONIC EPILEPSY |
| | ASSOCIATED WITH RAGGE . . . |
| | (OMIM:545000), MITOCHONDRIAL |
| | MYOPATHY, ENCEPHALOPATHY, . . . |
| | (OMIM:540000) |
| TRNL1 | MATERNALLY-INHERITED DIABETES |
| (4567) | AND DEAFNE . . . (ORPHA:225), MERRF |
| | (ORPHA:551), KEARNS-SAYRE SYNDROME |
| | (ORPHA:480), MELAS (ORPHA:550), |
| | MYOCLONIC EPILEPSY ASSOCIATED |
| | WITH RAGGE . . . (OMIM:545000), |
| | MITOCHONDRIAL MYOPATHY, |
| | ENCEPHALOPATHY, . . . (OMIM:540000) |
| PRKG1 | AORTIC ANEURYSM, FAMILIAL THORACIC |
| (5592) | 8 (OMIM:615436), FAMILIAL THORACIC |
| | AORTIC ANEURYSM AND AO . . . (ORPHA:91387) |
| C8ORF37 | RETINITIS PIGMENTOSA (ORPHA:791), |
| (157657) | RETINITIS PIGMENTOSA (OMIM:268000), |
| | CONE-ROD DYSTROPHY 16 (OMIM:614500), |
| | BARDET-BIEDL SYNDROME |
| | (ORPHA:110), CONE ROD DYSTROPHY |
| | (ORPHA:1872) |
| TRNQ | MERRF (ORPHA:551), MELAS (ORPHA:550), |
| (4572) | MITOCHONDRIAL MYOPATHY, |
| | ENCEPHALOPATHY, . . . (OMIM:540000) |
| TRNS1 | MERRF (ORPHA:551), DEAFNESS, |
| (4574) | AMINOGLYCOSIDE-INDUCED (OMIM:580000), |
| | MELAS (ORPHA:550), MITOCHONDRIAL |
| | MYOPATHY, ENCEPHALOPATHY, . . . |
| | (OMIM:540000) |
| TRNS2 | MERRF (ORPHA:551), USHER SYNDROME |
| (4575) | TYPE 3 (ORPHA:231183), MELAS |
| | (ORPHA:550), MITOCHONDRIAL |
| | MYOPATHY, ENCEPHALOPATHY, . . . |
| | (OMIM:540000) |
| TRNV | MITOCHONDRIAL MYOPATHY, |
| (4577) | ENCEPHALOPATHY, . . . (OMIM:540000) |
| TRNW | MITOCHONDRIAL MYOPATHY |
| (4578) | (OMIM:251900), MELAS (ORPHA:550), |
| | MITOCHONDRIAL MYOPATHY, |
| | ENCEPHALOPATHY, . . . (OMIM:540000) |
| LYZ (4069) | AMYLOIDOSIS, FAMILIAL |
| | VISCERAL (OMIM:105200) |
| ENG (2022) | TELANGIECTASIA, HEREDITARY |
| | HEMORRHAGIC, . . . (OMIM:187300), HEREDITARY |
| | HEMORRHAGIC TELANGIECTASIA (ORPHA:774) |
| MUC1 | MEDULLARY CYSTIC KIDNEY |
| (4582) | DISEASE 1 (OMIM:174000) |
| BBS12 | BARDET-BIEDL SYNDROME 12 |
| (166379) | (OMIM:615989), BARDET-BIEDL SYNDROME |
| | (ORPHA:110) |
| G6PC | GLYCOGEN STORAGE DISEASE IA (OMIM:232200) |
| (2538) | |
| SLC37A4 | GLYCOGEN STORAGE DISEASE IB |
| (2542) | (OMIM:232220), GLYCOGEN STORAGE |
| | DISEASE IC (OMIM:232240) |
| TNPO3 | PRIMARY BILIARY CHOLANGITIS (ORPHA:186) |
| (23534) | |
| TBL2 | WILLIAMS SYNDROME (ORPHA:904) |
| (26608) | |
| EDA2R | X-LINKED HYPOHIDROTIC |
| (60401) | ECTODERMAL DYSPLAS . . . (ORPHA:181) |
| H19 | WILMS TUMOR 1 (OMIM:194070), |
| (283120) | SILVER-RUSSELL SYNDROME (OMIM:180860), |
| | ISOLATED HEMIHYPERPLASIA |
| | (ORPHA:2128), NEPHROBLASTOMA (ORPHA:654), |
| | BECKWITH-WIEDEMANN SYNDROME |
| | (OMIM:130650), MULTIPLE TUMOR- |
| | ASSOCIATED CHROMOSOME |
| | REG . . . (OMIM:194071) |
| SMAD3 | LOEYS-DIETZ SYNDROME, TYPE 3 |
| (4088) | (OMIM:613795), FAMILIAL THORACIC AORTIC |
| | ANEURYSM AND AO . . . (ORPHA:91387) |
| SMAD4 | JUVENILE POLYPOSIS SYNDROME |
| (4089) | (OMIM:174900), MYHRE SYNDROME |
| | (OMIM:139210), JUVENILE POLYPOSIS/ |
| | HEREDITARY HEMORRHAGI . . . |
| | (OMIM:175050), PANCREATIC CANCER |
| | (OMIM:260350), HEREDITARY |
| | HEMORRHAGIC TELANGIECTASIA (ORPHA:774) |
| CEP19 | MORBID OBESITY AND SPERMATOGENIC |
| (84984) | FAILURE (OMIM:615703) |
| INVS | NEPHRONOPHTHISIS 2 (OMIM:602088), |
| (27130) | SENIOR-LOKEN SYNDROME |
| | (ORPHA:3156) |
| PIGM | GLYCOSYLPHOSPHATIDYLINOSITOL |
| (93183) | DEFICIENCY (OMIM:610293) |

TABLE 2

| Entrez | |
|---|---|
| ID | Gene |
| 183 | AGT |
| 3630 | INS |
| 1636 | ACE |
| 5972 | REN |
| 1906 | EDN1 |
| 3952 | LEP |
| 4846 | NOS3 |
| 118 | ADD1 |
| 4878 | NPPA |
| 1585 | CYP11B2 |
| 283 | ANG |
| 154 | ADRB2 |
| 185 | AGTR1 |
| 9370 | ADIPOQ |
| 7124 | TNF |
| 29984 | RHOD |
| 4306 | NR3C2 |
| 3569 | IL6 |
| 5468 | PPARG |
| 659 | BMPR2 |
| 1909 | EDNRA |
| 65266 | WNK4 |
| 1401 | CRP |
| 3043 | HBB |
| 6403 | SELP |
| 387 | RHOA |
| 5267 | SERPINA4 |
| 5054 | SERPINE1 |
| 3162 | HMOX1 |
| 3265 | HRAS |
| 59272 | ACE2 |
| 2056 | EPO |
| 65125 | WNK1 |
| 5743 | PTGS2 |
| 4843 | NOS2 |
| 7422 | VEGFA |
| 2147 | F2 |
| 3291 | HSD11B2 |
| 4018 | LPA |
| 348 | APOE |
| 6446 | SGK1 |
| 27430 | MAT2B |
| 6296 | ACSM3 |
| 3479 | IGF1 |
| 72 | ACTG2 |
| 133 | ADM |
| 338 | APOB |
| 2784 | GNB3 |
| 7450 | VWF |
| 4318 | MMP9 |
| 5465 | PPARA |
| 5443 | POMC |
| 5741 | PTH |
| 151 | ADRA2B |
| 3383 | ICAM1 |
| 4842 | NOS1 |
| 4023 | LPL |
| 1910 | EDNRB |
| 5530 | PPP3CA |
| 207 | AKT1 |
| 5617 | PRL |
| 847 | CAT |
| 857 | CAV1 |
| 51738 | GHRL |
| 5578 | PRKCA |
| 155 | ADRB3 |
| 3643 | INSR |
| 23327 | NEDD4L |
| 4790 | NFKB1 |
| 2353 | FOS |
| 4879 | NPPB |
| 2702 | GJA5 |
| 3553 | IL1B |
| 6532 | SLC6A4 |
| 3111 | HLA-DOA |
| 7056 | THBD |
| 6557 | SLC12A1 |
| 153 | ADRB1 |
| 7412 | VCAM1 |
| 5997 | RGS2 |
| 551 | AVP |
| 8862 | APLN |
| 1577 | CYP3A5 |
| 2006 | ELN |
| 796 | CALCA |
| 2868 | GRK4 |
| 6093 | ROCK1 |
| 1471 | CST3 |
| 6338 | SCNN1B |
| 1908 | EDN3 |
| 3091 | HIF1A |
| 284 | ANGPT1 |
| 7054 | TH |
| 150 | ADRA2A |
| 23564 | DDAH2 |
| 4524 | MTHFR |
| 3308 | HSPA4 |
| 948 | CD36 |
| 3375 | IAPP |
| 1956 | EGFR |
| 718 | C3 |
| 4313 | MMP2 |
| 6548 | SLC9A1 |
| 10911 | UTS2 |
| 476 | ATP1A1 |
| 2641 | GCG |
| 3290 | HSD11B1 |
| 1215 | CMA1 |
| 4803 | NGF |
| 2099 | ESR1 |
| 3240 | HP |
| 2100 | ESR2 |
| 186 | AGTR2 |
| 6647 | SOD1 |
| 2053 | EPHX2 |
| 136319 | MTPN |
| 2729 | GCLC |
| 6347 | CCL2 |
| 5879 | RAC1 |
| 1535 | CYBA |
| 3725 | JUN |
| 3949 | LDLR |
| 22796 | COG2 |
| 801 | CALM1 |
| 367 | AR |
| 4159 | MC3R |
| 6649 | SOD3 |
| 3953 | LEPR |
| 2944 | GSTM1 |
| 25801 | GCA |
| 7133 | TNFRSF1B |
| 3767 | KCNJ11 |
| 6750 | SST |
| 10891 | PPARGC1A |
| 56729 | RETN |
| 213 | ALB |
| 624 | BDKRB2 |
| 27347 | STK39 |
| 2908 | NR3C1 |
| 1621 | DBH |
| 1392 | CRH |
| 1565 | CYP2D6 |
| 4883 | NPR3 |
| 1584 | CYP11B1 |
| 4852 | NPY |
| 1536 | CYBB |
| 1113 | CHGA |
| 285 | ANGPT2 |
| 6648 | SOD2 |
| 2626 | GATA4 |
| 6337 | SCNN1A |
| 3791 | KDR |
| 6340 | SCNN1G |
| 4627 | MYH9 |
| 3156 | HMGCR |
| 5409 | PNMT |
| 5310 | PKD1 |
| 654 | BMP6 |
| 8654 | PDE5A |
| 1573 | CYP2J2 |
| 2697 | GJA1 |
| 2952 | GSTT1 |
| 5742 | PTGS1 |
| 920 | CD4 |
| 836 | CASP3 |
| 94 | ACVRL1 |
| 5327 | PLAT |
| 1188 | CLCNKB |
| 217 | ALDH2 |
| 3356 | HTR2A |
| 6352 | CCL5 |
| 6517 | SLC2A4 |
| 156 | ADRBK1 |
| 3667 | IRS1 |
| 5172 | SLC26A4 |
| 7040 | TGFB1 |
| 1432 | MAPK14 |
| 7351 | UCP2 |
| 147 | ADRA1B |
| 7432 | VIP |
| 1489 | CTF1 |
| 4312 | MMP1 |
| 2688 | GH1 |
| 335 | APOA1 |
| 649 | BMP1 |
| 6546 | SLC8A1 |
| 4881 | NPR1 |
| 1579 | CYP4A11 |
| 1576 | CYP3A4 |
| 6518 | SLC2A5 |
| 5243 | ABCB1 |
| 653361 | NCF1 |
| 2187 | FANCB |
| 717 | C2 |
| 3039 | HBA1 |
| 11132 | CAPN10 |
| 947 | CD34 |
| 5473 | PPBP |
| 3741 | KCNA5 |
| 3350 | HTR1A |
| 4868 | NPHS1 |
| 3684 | ITGAM |
| 55811 | ADCY10 |
| 2778 | GNAS |
| 3123 | HLA-DRB1 |
| 27035 | NOX1 |
| 1029 | CDKN2A |
| 6559 | SLC12A3 |
| 831 | CAST |
| 146 | ADRA1D |
| 8529 | CYP4F2 |
| 3818 | KLKB1 |
| 3636 | INPPL1 |
| 2153 | F5 |
| 7294 | TXK |
| 7941 | PLA2G7 |
| 2638 | GC |
| 3784 | KCNQ1 |
| 2869 | GRK5 |
| 6774 | STAT3 |
| 50507 | NOX4 |
| 3303 | HSPA1A |
| 6736 | SRY |
| 5333 | PLCD1 |
| 3320 | HSP90AA1 |
| 7369 | UMOD |
| 5179 | PENK |
| 404677 | CIMT |
| 4254 | KITLG |
| 2876 | GPX1 |
| 6582 | SLC22A2 |
| 6550 | SLC9A3 |
| 1812 | DRD1 |
| 2642 | GCGR |
| 7076 | TIMP1 |
| 7077 | TIMP2 |
| 5155 | PDGFB |
| 8490 | RGS5 |
| 1889 | ECE1 |
| 2200 | FBN1 |
| 567 | B2M |
| 51083 | GAL |
| 6530 | SLC6A2 |
| 3586 | IL10 |
| 5444 | PON1 |
| 7200 | TRH |
| 1907 | EDN2 |
| 490 | ATP2B1 |
| 119 | ADD2 |
| 3673 | ITGA2 |
| 135 | ADORA2A |
| 5624 | PROC |
| 5020 | OXT |
| 6271 | S100A1 |
| 720 | C4A |
| 4775 | NFATC3 |
| 1586 | CYP17A1 |
| 93649 | MYOCD |
| 516 | ATP5G1 |
| 1813 | DRD2 |
| 2250 | FGF5 |
| 51327 | AHSP |
| 55328 | RNLS |
| 5265 | SERPINA1 |
| 6696 | SPP1 |
| 7442 | TRPV1 |
| 5740 | PTGIS |
| 11117 | EMILIN1 |
| 5594 | MAPK1 |
| 10699 | CORIN |
| 4088 | SMAD3 |
| 7423 | VEGFB |
| 1588 | CYP19A1 |
| 535 | ATP6V0A1 |
| 64689 | GORASP1 |
| 652 | BMP4 |
| 6376 | CX3CL1 |
| 2770 | GNAI1 |
| 3827 | KNG1 |
| 2691 | GHRH |
| 355 | FAS |
| 9261 | MAPKAPK2 |
| 728 | C5AR1 |
| 596 | BCL2 |
| 1545 | CYP1B1 |
| 3758 | KCNJ1 |
| 3716 | JAK1 |
| 2597 | GAPDH |
| 358 | AQP1 |
| 23175 | LPIN1 |
| 7046 | TGFBR1 |
| 5592 | PRKG1 |
| 3990 | LIPC |
| 3082 | HGF |
| 23576 | DDAH1 |
| 3717 | JAK2 |
| 4973 | OLR1 |
| 3605 | IL17A |
| 488 | ATP2A2 |
| 196 | AHR |
| 5547 | PRCP |
| 4886 | NPY1R |
| 3075 | CFH |
| 5973 | RENBP |
| 64167 | ERAP2 |
| 1191 | CLU |
| 10550 | ARL6IP5 |
| 3572 | IL6ST |
| 1285 | COL4A3 |
| 3958 | LGALS3 |
| 9990 | SLC12A6 |
| 2247 | FGF2 |
| 7201 | TRHR |
| 3458 | IFNG |
| 650 | BMP2 |
| 328 | APEX1 |
| 6010 | RHO |
| 6275 | S100A4 |
| 8195 | MKKS |
| 9475 | ROCK2 |
| 7099 | TLR4 |
| 4663 | NA |
| 57142 | RTN4 |
| 6513 | SLC2A1 |
| 7043 | TGFB3 |
| 4548 | MTR |
| 3480 | IGF1R |
| 775 | CACNA1C |
| 481 | ATP1B1 |
| 6869 | TACR1 |
| 3559 | IL2RA |
| 6916 | TBXAS1 |
| 10159 | ATP6AP2 |
| 5627 | PROS1 |
| 50848 | F11R |
| 3576 | IL8 |
| 3297 | HSF1 |
| 6714 | SRC |
| 5216 | PFN1 |
| 148 | ADRA1A |
| 3119 | HLA-DQB1 |
| 1361 | CPB2 |
| 6817 | SULT1A1 |
| 6013 | RLN1 |
| 5652 | PRSS8 |
| 1559 | CYP2C9 |
| 1991 | ELANE |
| 2947 | GSTM3 |
| 2627 | GATA6 |
| 7830 | PHA2A |
| 4734 | NEDD4 |
| 4512 | COX1 |
| 65268 | WNK2 |
| 4129 | MAOB |
| 4609 | MYC |
| 3118 | HLA-DQA2 |
| 6277 | S100A6 |
| 27349 | MCAT |
| 3783 | KCNN4 |
| 64132 | XYLT2 |
| 9360 | PPIG |
| 4773 | NFATC2 |
| 23411 | SIRT1 |
| 3683 | ITGAL |
| 187 | APLNR |
| 967 | CD63 |
| 6927 | HNF1A |
| 11331 | PHB2 |
| 27345 | KCNMB4 |
| 5053 | PAH |
| 3329 | HSPD1 |
| 5595 | MAPK3 |
| 3371 | TNC |
| 2690 | GHR |
| 3115 | HLA-DPB1 |
| 6772 | STAT1 |
| 359 | AQP2 |
| 6523 | SLC5A1 |
| 4888 | NPY6R |
| 2981 | GUCA2B |
| 1667 | DEFA1 |
| 3779 | KCNMB1 |
| 1594 | CYP27B1 |
| 3397 | ID1 |
| 120 | ADD3 |
| 28893 | IGKV1D-39 |
| 2739 | GLO1 |
| 5029 | P2RY2 |
| 84432 | PROK1 |
| 2034 | EPAS1 |
| 3163 | HMOX2 |
| 12 | SERPINA3 |
| 3589 | IL11 |
| 3816 | KLK1 |
| 152 | ADRA2C |
| 5111 | PCNA |
| 6915 | TBXA2R |
| 356 | FASLG |
| 65010 | SLC26A6 |
| 2538 | G6PC |
| 22834 | ZNF652 |
| 3283 | HSD3B1 |
| 290 | ANPEP |
| 84106 | PRAM1 |
| 4000 | LMNA |
| 8089 | YEATS4 |
| 3598 | IL13RA2 |
| 240 | ALOX5 |
| 7293 | TNFRSF4 |
| 54957 | TXNL4B |
| 3776 | KCNK2 |
| 3574 | IL7 |
| 3579 | CXCR2 |
| 27177 | IL1F8 |
| 8942 | KYNU |
| 6668 | SP2 |
| 8435 | SOAT2 |
| 5563 | PRKAA2 |
| 1493 | CTLA4 |
| 1030 | CDKN2B |
| 3565 | IL4 |
| 84059 | GPR98 |
| 595 | CCND1 |
| 10267 | RAMP1 |
| 3182 | HNRNPAB |
| 10223 | GPA33 |
| 1649 | DDIT3 |
| 4683 | NBN |
| 3060 | HCRT |
| 2643 | GCH1 |
| 1815 | DRD4 |
| 1551 | CYP3A7 |
| 8842 | PROM1 |
| 2172 | FABP6 |
| 3117 | HLA-DQA1 |
| 2323 | FLT3LG |
| 84894 | LINGO1 |
| 361 | AQP4 |
| 5132 | PDC |
| 19 | ABCA1 |
| 7857 | SCG2 |
| 1870 | E2F2 |
| 5045 | FURIN |
| 197 | AHSG |
| 5226 | PGD |
| 10266 | RAMP2 |
| 613 | BCR |
| 10162 | LPCAT3 |
| 5894 | RAF1 |
| 9368 | SLC9A3R1 |
| 1363 | CPE |
| 5739 | PTGIR |
| 3606 | IL18 |
| 4010 | LMX1B |
| 2152 | F3 |
| 5539 | PPY |
| 2559 | GABRA6 |
| 9351 | SLC9A3R2 |
| 2280 | FKBP1A |
| 56606 | SLC2A9 |
| 858 | CAV2 |
| 5979 | RET |
| 5170 | PDPK1 |
| 3577 | CXCR1 |
| 1543 | CYP1A1 |
| 7398 | USP1 |
| 7057 | THBS1 |
| 8542 | APOL1 |
| 5460 | POU5F1 |
| 64240 | ABCG5 |
| 4205 | MEF2A |
| 7852 | CXCR4 |
| 1958 | EGR1 |
| 7052 | TGM2 |
| 10399 | GNB2L1 |
| 54331 | GNG2 |
| 1163 | CKS1B |
| 5445 | PON2 |
| 11200 | CHEK2 |
| 925 | CD8A |
| 7428 | VHL |
| 2668 | GDNF |
| 3316 | HSPB2 |
| 7349 | UCN |
| 140628 | GATA5 |
| 6514 | SLC2A2 |
| 552 | AVPR1A |
| 157 | ADRBK2 |
| 6525 | SMTN |
| 3069 | HDLBP |
| 10615 | SPAG5 |
| 128 | ADH5 |
| 662 | BNIP1 |
| 4760 | NEUROD1 |
| 930 | CD19 |
| 2768 | GNA12 |
| 4887 | NPY2R |
| 406983 | MIR200A |
| 3066 | HDAC2 |
| 3439 | IFNA1 |
| 7048 | TGFBR2 |
| 2166 | FAAH |
| 6402 | SELL |
| 4907 | NT5E |
| 760 | CA2 |
| 3491 | CYR61 |
| 5698 | PSMB9 |
| 4880 | NPPC |
| 200316 | APOBEC3F |
| 54583 | EGLN1 |
| 5604 | MAP2K1 |
| 5340 | PLG |
| 241 | ALOX5AP |
| 6279 | S100A8 |
| 84666 | RETNLB |
| 9927 | MFN2 |
| 2011 | MARK2 |
| 26548 | ITGB1BP2 |
| 7295 | TXN |
| 276 | AMY1A |
| 4160 | MC4R |
| 345 | APOC3 |
| 5058 | PAK1 |
| 64131 | XYLT1 |
| 7421 | VDR |
| 1581 | CYP7A1 |
| 2678 | GGT1 |
| 914 | CD2 |
| 7536 | SF1 |
| 9099 | USP2 |
| 4314 | MMP3 |
| 23560 | GTPBP4 |
| 8801 | SUCLG2 |
| 29110 | TBK1 |
| 1832 | DSP |
| 5175 | PECAM1 |
| 7018 | TF |
| 623 | BDKRB1 |
| 4287 | ATXN3 |
| 2870 | GRK6 |
| 6464 | SHC1 |
| 1407 | CRY1 |
| 1901 | S1PR1 |
| 598 | BCL2L1 |
| 525 | ATP6V1B1 |
| 9138 | ARHGEF1 |
| 10935 | PRDX3 |
| 3600 | IL15 |
| 8601 | RGS20 |
| 2735 | GLI1 |
| 10268 | RAMP3 |
| 1312 | COMT |
| 2057 | EPOR |
| 4323 | MMP14 |
| 3957 | LGALS2 |
| 6262 | RYR2 |
| 2550 | GABBR1 |
| 51179 | HAO2 |
| 5329 | PLAUR |
| 477 | ATP1A2 |
| 5251 | PHEX |
| 783 | CACNB2 |
| 113026 | PLCD3 |
| 1445 | CSK |
| 7060 | THBS4 |
| 2033 | EP300 |
| 4137 | MAPT |
| 56670 | SUCNR1 |
| 140803 | TRPM6 |
| 1356 | CP |
| 1278 | COL1A2 |
| 1827 | RCAN1 |
| 5601 | MAPK9 |
| 8840 | WISP1 |
| 4360 | MRC1 |
| 10328 | COX4NB |
| 5582 | PRKCG |
| 1026 | CDKN1A |
| 5770 | PTPN1 |
| 3554 | IL1R1 |
| 11012 | KLK11 |
| 4086 | SMAD1 |
| 776 | CACNA1D |
| 841 | CASP8 |
| 134 | ADORA1 |
| 25797 | QPCT |
| 202333 | CMYA5 |
| 2022 | ENG |
| 1 | A1BG |
| 64241 | ABCG8 |
| 6098 | ROS1 |
| 8797 | TNFRSF10A |
| 3241 | HPCAL1 |
| 7827 | NPHS2 |
| 5550 | PREP |
| 1756 | DMD |
| 9294 | S1PR2 |
| 10568 | SLC34A2 |
| 57105 | CYSLTR2 |
| 4316 | MMP7 |
| 3202 | HOXA5 |
| 3557 | IL1RN |
| 7067 | THRA |
| 3105 | HLA-A |
| 558 | AXL |
| 117584 | RFFL |
| 57561 | ARRDC3 |
| 28 | ABO |
| 2949 | GSTM5 |
| 4889 | NPY5R |
| 9982 | FGFBP1 |
| 2701 | GJA4 |
| 8912 | CACNA1H |
| 1814 | DRD3 |
| 554 | AVPR2 |
| 80310 | PDGFD |
| 2539 | G6PD |
| 2492 | FSHR |
| 8879 | SGPL1 |
| 3484 | IGFBP1 |
| 27129 | HSPB7 |
| 7039 | TGFA |
| 1557 | CYP2C19 |
| 10580 | SORBS1 |
| 8972 | MGAM |
| 79924 | ADM2 |
| 4317 | MMP8 |
| 5562 | PRKAA1 |
| 3196 | TLX2 |
| 6011 | GRK1 |
| 21 | ABCA3 |
| 1558 | CYP2C8 |
| 4509 | ATP8 |
| 7038 | TG |
| 3676 | ITGA4 |
| 8170 | SLC14A2 |
| 5139 | PDE3A |
| 23641 | LDOC1 |
| 9061 | PAPSS1 |
| 246 | ALOX15 |
| 149420 | PDIK1L |
| 246734 | NPCDR1 |
| 6197 | RPS6KA3 |
| 8671 | SLC4A4 |
| 5919 | RARRES2 |
| 4092 | SMAD7 |
| 5144 | PDE4D |
| 23365 | ARHGEF12 |
| 3312 | HSPA8 |
| 10153 | CEBPZ |
| 640 | BLK |
| 1773 | DNASE1 |
| 6522 | SLC4A2 |
| 7225 | TRPC6 |
| 3274 | HRH2 |
| 799 | CALCR |
| 2705 | GJB1 |
| 1012 | CDH13 |
| 283120 | H19 |
| 6581 | SLC22A3 |
| 1236 | CCR7 |
| 4087 | SMAD2 |
| 1791 | DNTT |
| 2997 | GYS1 |
| 5328 | PLAU |
| 5800 | PTPRO |
| 3481 | IGF2 |
| 1803 | DPP4 |
| 5055 | SERPINB2 |
| 5687 | PSMA6 |
| 4982 | TNFRSF11B |
| 7498 | XDH |
| 1583 | CYP11A1 |
| 6786 | STIM1 |
| 3611 | ILK |
| 1013 | CDH15 |
| 1234 | CCR5 |
| 64478 | CSMD1 |
| 6536 | SLC6A9 |
| 3077 | HFE |
| 23523 | CABIN1 |
| 36 | ACADSB |
| 57154 | SMURF1 |
| 4659 | PPP1R12A |
| 4718 | NDUFC2 |
| 3552 | IL1A |
| 5319 | PLA2G1B |
| 116985 | ARAP1 |
| 83990 | BRIP1 |
| 9971 | NR1H4 |
| 4358 | MPV17 |
| 3551 | IKBKB |
| 3753 | KCNE1 |
| 7410 | VAV2 |
| 7350 | UCP1 |
| 5270 | SERPINE2 |
| 51477 | ISYNA1 |
| 81631 | MAP1LC3B |
| 1395 | CRHR2 |
| 6558 | SLC12A2 |
| 3745 | KCNB1 |
| 1589 | CYP21A2 |
| 3627 | CXCL10 |
| 90459 | ERI1 |
| 5159 | PDGFRB |
| 3814 | KISS1 |
| 585 | BBS4 |
| 466 | ATF1 |
| 2922 | GRP |
| 2752 | GLUL |
| 2170 | FABP3 |
| 6401 | SELE |
| 5292 | PIM1 |
| 1950 | EGF |
| 9607 | CARTPT |
| 112399 | EGLN3 |
| 167227 | DCP2 |
| 8518 | IKBKAP |
| 3782 | KCNN3 |
| 25828 | TXN2 |
| 7166 | TPH1 |
| 7352 | UCP3 |
| 1490 | CTGF |
| 55824 | PAG1 |
| 818 | CAMK2G |
| 3558 | IL2 |
| 5338 | PLD2 |
| 8851 | CDK5R1 |
| 7137 | TNNI3 |
| 26503 | SLC17A5 |
| 6890 | TAP1 |
| 406947 | MIR155 |
| 94274 | PPP1R14A |
| 3596 | IL13 |
| 4763 | NF1 |
| 11120 | BTN2A1 |
| 7037 | TFRC |
| 6863 | TAC1 |
| 5184 | PEPD |
| 6667 | SP1 |
| 64663 | SPANXC |
| 7224 | TRPC5 |
| 6566 | SLC16A1 |
| 5069 | PAPPA |
| 25824 | PRDX5 |
| 8856 | NR1I2 |
| 301 | ANXA1 |
| 2848 | GPR25 |
| 4653 | MYOC |
| 4142 | MAS1 |
| 3248 | HPGD |
| 842 | CASP9 |
| 6387 | CXCL12 |
| 38 | ACAT1 |
| 1282 | COL4A1 |
| 5728 | PTEN |
| 4345 | CD200 |
| 5173 | PDYN |
| 4143 | MAT1A |
| 144100 | PLEKHA7 |
| 1508 | CTSB |
| 3357 | HTR2B |
| 966 | CD59 |
| 11093 | ADAMTS13 |
| 4929 | NR4A2 |
| 5071 | PARK2 |
| 54602 | NDFIP2 |
| 9518 | GDF15 |
| 7490 | WT1 |
| 116285 | ACSM1 |
| 3609 | ILF3 |
| 43 | ACHE |
| 66036 | MTMR9 |
| 192115 | MA |
| 9575 | CLOCK |
| 8876 | VNN1 |
| 6521 | SLC4A1 |
| 221935 | SDK1 |
| 5744 | PTHLH |
| 4223 | MEOX2 |
| 5295 | PIK3R1 |
| 5534 | PPP3R1 |
| 121278 | TPH2 |
| 250 | ALPP |
| 239 | ALOX12 |
| 6374 | CXCL5 |
| 4012 | LNPEP |
| 1387 | CREBBP |
| 88 | ACTN2 |
| 52 | ACP1 |
| 9630 | GNA14 |
| 3766 | KCNJ10 |
| 2169 | FABP2 |
| 3106 | HLA-B |
| 3597 | IL13RA1 |
| 1544 | CYP1A2 |
| 4855 | NOTCH4 |
| 6326 | SCN2A |
| 142 | PARP1 |
| 51726 | DNAJB11 |
| 54795 | TRPM4 |
| 6332 | SCN7A |
| 1182 | CLCN3 |
| 6862 | T |
| 1071 | CETP |
| 2328 | FMO3 |
| 4684 | NCAM1 |
| 5982 | RFC2 |
| 123041 | SLC24A4 |
| 2523 | FUT1 |
| 2052 | EPHX1 |
| 2335 | FN1 |
| 890 | CCNA2 |
| 1634 | DCN |
| 7431 | VIM |
| 4914 | NTRK1 |
| 9498 | SLC4A8 |
| 351 | APP |
| 1003 | CDH5 |
| 5046 | PCSK6 |
| 4793 | NFKBIB |
| 6368 | CCL23 |
| 2932 | GSK3B |
| 5396 | PRRX1 |
| 4481 | MSR1 |
| 9910 | RABGAP1L |
| 10052 | GJC1 |
| 27063 | ANKRD1 |
| 866 | SERPINA6 |
| 9356 | SLC22A6 |
| 3485 | IGFBP2 |
| 4586 | MUC5AC |
| 84897 | TBRG1 |
| 7528 | YY1 |
| 5167 | ENPP1 |
| 8870 | IER3 |
| 406952 | MIR17 |

1. A method of identifying genes associated with poor clinical outcomes for a particular cancer, comprising a cohort of patients with the said cancer, identifying at least one comorbid medical condition, determining the gene alterations associated with at least one comorbidity, determining the gene expression level associated with at least one comorbidity, normalizing said gene expression level against the expression level of a reference set of RNA transcripts in patients without the cancer or comorbidity, performing a statistical analysis comparing the pathological gene expression level with normal gene expression level, and creating a database of statistically significant genes wherein the expression level of said genes encoding a comorbidity are associated with poor clinical outcomes for the particular cancer, and wherein an outcome for the cancer can be graded from the expression of genes in the database.

2. The method of claim 1 wherein the groups may comprise cancer in different stages grouped into two or more groups.

3. The method of claim 1, wherein the comorbidity is selected from one or more of essential hypertension, obesity, diabetes type 1, diabetes type 2, metabolic syndrome, endocrinopathies, chronic obstructive pulmonary disease, chronic kidney disease, coronary artery disease, stroke, depression, dysthymia, anxiety disorders, bipolar disorders, drug abuse, alcohol abuse, smoking Parkinson's Disease, Alzheimer's Disease.

4. The method of claim 1 wherein the genes include one or more of the genes listed in either table 1 or table 2.

5. The method of claim 1 wherein the gene alteration and gene expression is quantified.

6. The method of claim 1 wherein the genes identified as significant are further assessed for their relevance to oncogenes.

7. A method of treating cancer in a patient suffering from cancer by treating abnormalities in at least one gene encoding a comorbidity associated with a particular cancer, comprising identifying genes encoding a comorbidity associated with a poor clinical outcomes for a particular cancer, identifying a cohort of patients with the said cancer, identifying at least one comorbid medical condition, determining the gene alterations associated with the at least one comorbidities, determining the gene expression level associated with the said comorbidity, normalizing said gene expression level against the expression level of a reference set of RNA transcripts in patients without the cancer or comorbidity, performing a statistical analysis comparing the pathological gene expression level with normal gene expression level, creating a database of statistically significant genes wherein the expression level of said genes is negatively associated with worse outcomes for the particular cancer, and treating the cancer by prescribing therapies that inhibit the expression of said genes.

8. The method of claim 7, wherein the treatment comprises a treatment selected from surgery, radiation, chemotherapy, watchful waiting, active surveillance, immunotherapy, thermotherapy, embolization and cryotherapy.

9. The method of claim 7 wherein the genes and their products may be blocked by using a suitable drug thereby achieving either a cure, or a delay in progression of cancer.

10. The method of claim 7, wherein the comorbidity is hypertension.

11. A method of treating cancer in a patient suffering from cancer by treating abnormalities in at least one gene encoding a comorbidity associated with a particular cancer, comprising
a. selecting a cohort of patients suffering with the particular cancer;
b. identifying a subset of patients having comorbidities;
c. dividing the cohort into a training set of patients of approximately two-thirds of the cohort, and a validation set of patients of approximately one-third of the cohort;
d. further stratify the training set stratify the training set by factors associated with cancer propensity;
e. identifying genes encoding comorbidities in the training set;
f. identifying mutations, alterations, or differential gene expression in the genes encoding comorbidities in each member of the training set and normalize the gene expression level against the reference set in patients without the cancer or comorbidity;
g. correlating mutations in the genes encoding comorbidities in the training set with the severity of the cancer for each member of the training set by determining the gene expression level associated with each comorbidity and normalizing the gene expression level against the expression level of a reference set of RNA transcripts in patients without the cancer or comorbidity;
h. applying the correlations from step (g) to the validation set and determining if the correlations are statistically significant to cancer severity in the validation set;
i. if the results from step (h) are statistically significant, then apply the analysis to a further one or more patients not in the cohort; and
j. treating the cancer by prescribing therapies that inhibit the expression of the one or more genes encoding comorbidities.

12. The method of claim 11, wherein the division of the cohort is by randomly assigning patients into each of the sets.

13. The method of claim 11, wherein the cohort is further stratified or based on one or more factors associated with cancer propensity.

14. The method of claim 11 wherein the membership of training set and validation set are shuffled after step (g), and repeating the analysis from step (e).

15. The method claim 14 wherein the analysis from step (e) is repeated two or more times.
